(19) United States
(12) Patent Application Publication
(10) Pub. No.: US 2011/0300154 A1
(43) Pub. Date: Dec. 8, 2011

(54) TREATMENT OF AIRWAY HYPERREACTIVITY

(75) Inventors: Dale T. Umetsu, Newton, MA (US); Muriel Pichavant, Mons-en-Baroeul (FR); Rosemarie Helena DeKruyff-Umetsu, Newton, MA (US); Everett Hurteau Meyer, Redwood City, CA (US); Stephanie Ann Shore, Lexington, MA (US)

(73) Assignees: CHILDREN'S MEDICAL CENTER CORPORATION, Boston, MA (US); PRESIDENT AND FELLOWS OF HARVARD COLLEGE, Cambridge, MA (US); THE BOARD OF TRUSTEES OF THE LELAND STANFORD JUNIOR UNIVERSITY, Palto Alto, CA (US)

(21) Appl. No.: 12/674,191

(22) PCT Filed: Aug. 21, 2008
(86) PCT No.: PCT/US08/73811

§ 371 Date: Aug. 29, 2011

Related U.S. Application Data
(60) Provisional application No. 60/957,011, filed on Aug. 21, 2007.

(51) Int. Cl. A61K 39/395; A61K 31/7088; A61P 11/06; A61K 31/7028; A61P 37/06; A61K 38/17; A61K 31/713

(52) U.S. Cl. 424/158.1; 514/1.7; 424/172.1; 514/44 A; 424/184.1

(57) ABSTRACT

The invention provides strategies for treating and preventing airway hyperreactivity and non-allergic asthma comprising antagonizing IL-17 activity and/or production by iNKT cells. Provided herein is a method of diagnosing non-allergic asthma and airway hyperreactivity comprising neutrophils quantification in sputum.

### CROSS REFERENCE TO RELATED APPLICATION

This application claims benefit under 35 U.S.C. §119(e) of the U.S. provisional application No. 60/957,011 filed Aug. 21, 2007, the contents of which are incorporated herein by reference in its entirety.

### GOVERNMENT SUPPORT

The invention was made with Government support under grant HL062348 and AI054456 from the National Institute of Health. The Government has certain rights in the invention.

### REFERENCE TO SEQUENCE LISTING AND COMPACT DISK

Applicants assert that the paper copy of the Sequence Listing is identical to the Sequence Listing in computer readable form found on the accompanying computer disk. Applicants incorporate the contents of the sequence listing by reference in its entirety.

### BACKGROUND OF THE INVENTION

Airway hyperreactivity is defined as the narrowing of stimulated air passages, having a tendency to sudden narrowing of the air passages of the lungs in response to stimuli such as pollen grains in the air, changes of temperature, emotional shock, or exercise. Airway hyperreactivity (AHR) is a cardinal feature of asthma, and is observed in all forms of asthma, including asthma induced with allergen and non-allergen such as ozone exposure.

Bronchial asthma is a heterogeneous syndrome associated with diverse factors, including allergen sensitization, infection, obesity, as well as exposure to air pollution (1). Regardless of the trigger, asthma is associated with reversible airway obstruction and airway hyperreactivity (AHR), an increased sensitivity of the airways to nonspecific stimuli such as cold air or respiratory irritants, and quantitated by responsiveness to methacholine or histamine (2).

Diverse mechanisms have been proposed to explain the pathogenesis of various asthma phenotypes: allergen induced and non-allergen-induced, including allergic inflammation, Th2 cells, eosinophils, basophils 3, neutrophils 4, and oxidative stress (5). Thus, different forms of asthma appear to depend on distinct cell types and pathways. In addition, the factors and mechanisms that determine the occurrence of airway hyperreactivity in the general population are not clearly understood.

Now in the field of asthma and AHR, exposure to ozone, a major component of air pollution produces a form of AHR not thought to involve immune mechanisms. Exposure to ozone induces a form of asthma that occurs in the absence of adaptive immunity, characterized by airway neutrophilia and not eosinophilia, but nevertheless associated with airway hyperreactivity (AHR), a cardinal feature of asthma. In children, ambient exposure to ozone increases asthmatic symptoms even at concentrations below the U.S. Environmental Protection Agency standard (20), and hospital admissions for asthma are higher on days of high ambient ozone concentrations (21, 22). Even in healthy individuals, exposure to ozone induces the development of AHR that is associated with airway epithelial cell damage and an increase in neutrophils and inflammatory mediators (TNFα, CXCL-8/IL-8, IL-6, GM-CSF) in proximal airways (23, 24), and asthmatics appear to be more susceptible to the adverse effects of this pollutant (25). Currently, there is still a need for a better understand the immune responses behind ozone-induced AHR. Because severe, non-allergic asthma is so poorly understood, there is very limited therapeutic options. Therefore, there is a need to develop effective therapeutics for treating severe, non-allergic asthma such as ozone-induced asthma and AHR.

### SUMMARY OF THE INVENTION

Airway hyperreactivity (AHR) and asthma can be divided into two large groups: allergen and non-allergen induced. Embodiments of the present invention is based on the discovery that iNKT cells and IL-17 production are required for AHR induced by repeated ozone exposure. With repeated exposure to ozone, the iNKT cells synthesized IL-17 and it is the Il-17 cytokine produced that illicit the characteristic features of non-allergic AHR, infiltration of neutrophils rather than eosinophils. Moreover, only in ozone-induced AHR, i.e. non-allergic AHR and asthma, that IL-17 is required. More importantly, treatment of mice with anti-IL-17 blocking antibody greatly reduced the ozone-induced AHR and IL-17 production in the lungs of mice. In addition, in the absence of ozone, direct activation of iNKT cells with α-GalactosylCeramide (α-GalCer) induces AHR in monkeys. Inhibiting IL-17 production, neutralizing Il-17, and/or inhibiting iNKT cell activation are potential therapeutic avenues for the treatment and/or prevention of allergic and non-allergic AHR and asthma.

Accordingly, provided herein is a method of treating non-allergic asthma in an individual comprising selecting an individual diagnosed with non-allergic asthma and administering to the individual an effective amount of an IL-17 antagonist. In one embodiment, the non-allergic asthma is ozone-induced asthma. Ozone exposure can lead to ozone-induced asthma and AHR that are associated with IL-17 production by pulmonary iNKT and T cells. By neutralizing the pro-inflamatory and chemotactic effects of IL-17 using an IL-17 antagonist such as anti-IL-17 antibodies, the development of ozone-induced but not allergen-induced AHR can be prevented. While iNKT cells are required for both allergen-induced as well as ozone-induced AHR, the pulmonary inflammation induced by ozone exposure is very distinct from that induced by allergen.

In one embodiment, the diagnosis of non-allergic asthma in an individual having non-allergic asthma is made by measuring the level of neutrophils in a sample of sputum from the individual. In one embodiment, if the neutrophil level is greater than 37% of the total cell count in the sputum sample, the individual is considered to have non-allergic asthma.

An IL-17 antagonist can be to any organic, inorganic molecule, or biologic that can block, inhibits, and prevents the cytokine IL-17A ligand-to-IL-17 receptor interaction and the consequent downstream signaling pathway. The IL-17 antagonist embodied in the methods described herein is selected from a group consisting of: a soluble form of an IL-17 receptor; an inhibitory anti-IL-17 antibody; an inhibitory anti-IL-17R antibody; a fusion protein containing IL-17 receptor; a fusion protein containing IL-17 binding fragment of IL-17 receptor; an IL-17 antagonistic small molecule; an antisense IL-17 nucleic acid molecule; an antisense IL-17 receptor nucleic acid molecule; an siRNA IL-17 nucleic acid molecule; and an siRNA IL-17 receptor nucleic acid molecule. A biologic is any preparation, such as a drug, a vaccine, protein, a protein fragment, an antibody, or an antitoxin, that is synthesized from living organisms or their products.

The present invention has shown that elevated level of pro-inflammatory cytokine IL-17 was produced by both iNKT cell and activated T-cells in the lungs. Accordingly, in one embodiment, the methods described herein further includes administering to an individual an effective amount of NKT cell inhibitory agent. The NKT inhibitory agent inhibits NKT cell immune function through interaction with an antigen presenting molecule. Exemplary of an NKT cell inhibitory agent is β-galactosylceramide.

Also embodied in the invention is a method of treating an individual at risk of non-allergic asthma. The methods includes, for example, measuring the level of neutrophils in a sample of sputum from an individual; administering to the individual an effective amount of an IL-17 antagonist when the level of neutrophils is above a predetermined level. The individual is considered at risk of non-allergic asthma if the individual has a previous non-allergic asthma attack and/or if the individual has an elevated percentage of neutrophils in the lung sputum.

### BRIEF DESCRIPTION OF THE DRAWINGS

FIG. 1. Ozone exposure induces AHR and increases airway inflammation in WT but not in CD1d^{-/-} mice. WT BALB/c mice and NKT cell deficient CD1d^{-/-} mice were exposed 3 times to 1 ppm of ozone over 5 days or to room air.

FIG. 1A. Changes in lung resistance (RL) were measured in anesthetized, tracheotomized, intubated and mechanically ventilated mice. Ozone exposure induces an increase in AHR in WT but not in CD1d^{-/-} mice.

FIG. 1B. WT BALB/c and CD1d^{-/-} mice were exposed to ozone as in FIG. 1A, and treated with anti-CD1d blocking mAb or isotype control mAb. Ozone-induced AHR was prevented by the anti-CD1d mAb treatment.

FIG. 1C. WT BALB/c and CD1d^{-/-} mice were exposed to ozone as in FIG. 1A, and treated with anti-CD1d blocking mAb or isotype control mAb. BAL fluid was collected 24 h after the last ozone challenge. Total and differential cell counts were evaluated in bronchoalveolar lavage (BAL) fluid. Ozone exposure induces pulmonary inflammation associated with neutrophils.

FIG. 1D. Ozone exposure increases iNKT cell number in BAL of WT but not CD1d^{-/-} mice. Anti-CD1d Ad treatment decreases α-GC-loaded CD1d Tetramer⁺ TCR13⁺ iNKT cell-infiltration after ozone exposure. Results are expressed as mean±SEM. p<0.05(*) and p<0.01 (**) compared to mice exposed to air. These results represent 1 out of 4 experiments with 5 mice in each group.

FIG. 2. Ozone exposure induces AHR in WT but not in Jα18^{-/-} mice. WT BALB/c and NKT cell deficient Jα18^{-/-} mice were exposed 3 times to 1 ppm of ozone versus air.

FIG. 2A. Changes in lung resistance (RL) were measured on anesthetized, tracheotomized, intubated and mechanically ventilated mice. Ozone exposure induces AHR in WT but not in Jα18^{-/-} mice.

FIG. 2B. Total and differential cell counts in BAL were evaluated.

FIG. 2C. Ozone exposure increases iNKT cell number in BAL of WT but not Jα18^{-/-} mice.

FIG. 2D. Adoptive transfer of WT iNKT cells into Jα18^{-/-} mice partially restores AHR induced by repeated ozone exposure. Results are expressed as mean±SEM. p<0.05(*) and p<0.01 (**) compared to mice exposed to air. These results represent 1 out of 4 experiments with 5 mice in each group.

FIG. 3. NKT cells and T cells produce IL-17 after ozone exposure. WT BALB/c mice were exposed 3 times to 1 ppm of ozone versus air. Intracellular IL-4, IFN-γ, IL-17, and IL-10 staining was performed on T cell-enriched lung cells without further stimulation, but treated with GolgiStop for 2 h.

FIG. 3A. iNKT cells were analyzed by gating on CD1d tetramer⁺ TCR13⁺ cells (top). T cells were analyzed by gating on CD1d tetramer⁻ cells (bottom). The flow cytometry data are provided as dot plots. Numbers in each quadrant indicate the percentage of cells in that quadrant. The number of dots (events) is much greater for T cells than for iNKT cells because the number of NKT cells in the lungs is only a fraction of the number of T cells present. Data are representative of three independent experiments.

FIG. 3B. MHC class II^{-/-} mice were exposed to ozone or air. Changes in lung resistance (RL) were measured on anesthetized, tracheotomized, intubated, and mechanically ventilated mice. Ozone exposure induced significant AHR in WT mice and MHC class II^{-/-} mice.

FIG. 3C. C57BL/6 mice were exposed to ozone. iNKT cells in the BAL fluid were analyzed by gating on CD1d tetramer⁺ TCR13⁺ cells, and by staining for expression of NK1.1 and IL-17. The IL-17⁺ cells were NK1.1 negative.

FIG. 4. IL-17 is involved in ozone-induced but not in OVA-induced AHR. (A and B): The response of WT mice to ozone is greatly reduced by treatment with anti-IL-17 mAb (aIL-17 Ab). WT mice were exposed 3 times to 1 ppm ozone versus air and treated with anti-IL-17 blocking mAb or isotype control.

FIG. 4A. Airway responsiveness to methacholine was measured in anesthetized, tracheotomized, intubated and mechanically ventilated mice and reported as increase in airway resistance.

FIG. 4B. BAL fluid was collected 24 h after the last airway challenge. Total and differential cell counts were evaluated. Treatment with anti-IL-17 mAb reduced airway lymphocytes and neutrophils.

FIG. 4C. Anti-IL-17 mAb treatment does not reduce OVA-induced AHR. Mice were sensitized i.p. and challenged intranasally with OVA for 3 d, and were treated with anti-IL-17 blocking mAb or isotype control 1 d before the 3 OVA challenges.

FIG. 4D. Anti-IL-17 mAb treatment does not reduce OVA-induced airway inflammation. Mice were sensitized i.p. and challenged intranasally with OVA for 3 d, and were treated with anti-IL-17 blocking mAb or isotype control 1 d before the 3 OVA challenges. BAL fluid was collected and evaluated as in B. Results are expressed as the mean±the SEM. P≦0.05 (*) and P≦0.01 (**) compared with untreated mice or mice exposed to air. Data are representative of two independent experiments with five mice in each group.

FIG. 4E. IL-17^{-/-} mice fail to develop ozone-induced AHR. Mice were treated as in FIGS. 4A and B, and examined for AHR.

FIG. 4F. IL-17^{-/-} mice develop normal OVA-induced AHR. IL-17^{-/-} mice were sensitized and challenged with OVA before examination for AHR by challenge with methacholine.

FIG. 5. IL-4 and IL-13 are involved in ozone-induced AHR and airway inflammation. WT BALB/c and IL-4^{-/-}/IL-13^{-/-} double knockout mice were exposed 3 times to 1 ppm of ozone versus air.

FIG. 5A. Airway responsiveness was measured as in FIG. 4A. IL-4^{-/-}/IL-13^{-/-} double knockout mice failed to develop ozone-induced AHR.

FIG. 5B. Total and differential cell counts were evaluated in BAL fluid. IL-4^{-/-}/IL-13^{-/-} double-knockout mice failed to develop ozone-induced airway inflammation.

FIG. 5C. Adoptive transfer of IL-4^{-/-}/IL-13^{-/-} iNKT cells into Jα18^{-/-} failed to restore ozone-induced AHR. Results are expressed as mean±SEM. p<0.05(*) and p<0.01 (**) compared to mice exposed to air, and double knockout mice. These results are representative of 3 experiments.

FIG. 5D. IL-13^{-/-} mice were exposed to ozone before measurement of AHR, as in FIG. 5A. AHR failed to occur in the IL-13^{-/-} mice.

FIG. 5E. IL-4^{-/-} mice were exposed to ozone before measurement of AHR, as in FIG. 5A. AHR failed to occur in the IL-4^{-/-} mice.

FIG. 6A. AHR was measured in each monkey after challenged with α-GalCer or vehicle. Challenge of monkeys with α-GalCer induces airway hyperreactivity. The percentage change in RL from baseline in response to increasing concentrations of methacholine is shown.

FIG. 6B. Combining data from four monkeys, α-GalCer induced a significantly higher percentage change in lung resistance (RL) from baseline compared to that with vehicle control (p=0.0003)

### DETAILED DESCRIPTION OF THE INVENTION

Invariant T cell receptor (TCR) natural killer T (iNKT) cells comprise a newly described, unique subset of lymphocytes that express features of both classical T cells and Natural Killer cells. iNKT cells express a conserved/invariant TCR repertoire consisting of Vα14-Jα18 (in mice) or Vα24-Jα18 (in humans) (6) and are activated by glycolipid antigens presented by the non polymorphic MHC class 1-like protein, CD1d, which is widely expressed by airway and intestinal epithelial cells, B cells, macrophages and dendritic cells. This indicates that iNKT cells play a pivotal role in immunity (7, 8). Activation of iNKT cells results in an innate-like immune response, with rapid production of large quantities of cytokines, including IL-4 and IFN-γ (6, 9), and this rapid response endows iNKT cells with the capacity to critically amplify adaptive immunity and regulate the development of polarized T cells. The rapid production of cytokines by iNKT cells has been shown to regulate the development of a number of diseases, including diabetes mellitus, colitis, autoimmune neurological disease, rejection of malignant tumors, infectious diseases, and asthma (10-14).

While iNKT cells definitely play a very important role in the development of asthma and AHR (13-19), it is still controversial as to the role of IL-17 play in asthma. IL-17A is a pro inflammatory cytokine that regulates both granulopoiesis and recruitment of neutrophils into sites of inflammation (Yao et al. (1995) J. Immunol. 155:5483-86; Ye et al. (2001) J. Exp. Med. 194:519-28; Kolls et al. (2003) Am. J. Respir. Cell Mol. Biol. 28:9-11; Laan et al. (1999) J. Immunol. 162:2347-52; Linden et al. (2000) Eur. Respir. J. 15:973-77). There are conflicting results regarding the role of IL-17A in asthma. IL-17A is known to play an important role in activating T cells (33), enhancing neutrophil accumulation and inducing IL-6, TNF-α and TGF-β production, which increase airway inflammation (34). Although IL-17A (protein and mRNA) is present in the lungs of patients with severe asthma along with eosinophils and neutrophils (35-37), or in the lungs of mice chronically exposed to ovalbulim (OVA) (38), other studies have indicated a potent inhibitory role for IL-17 in asthma (39).

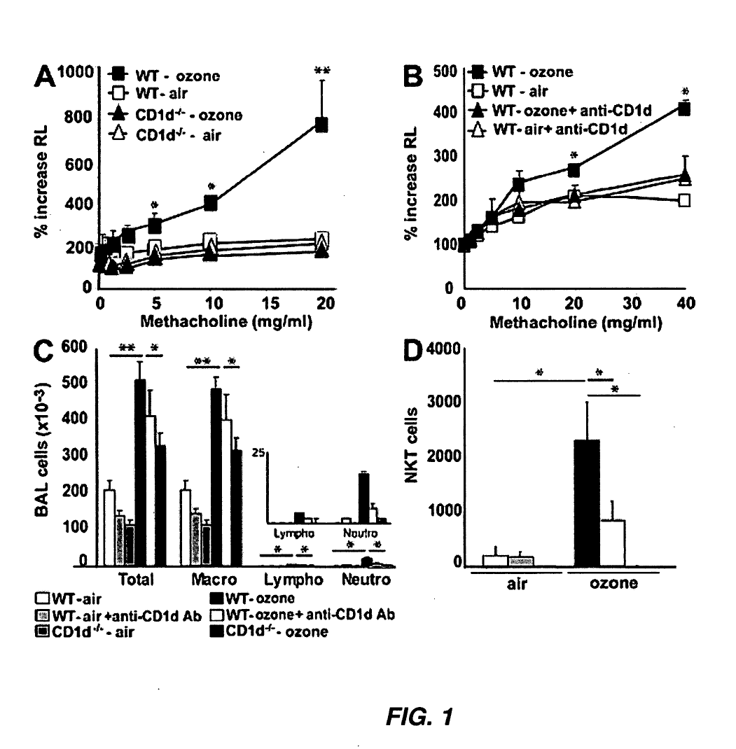

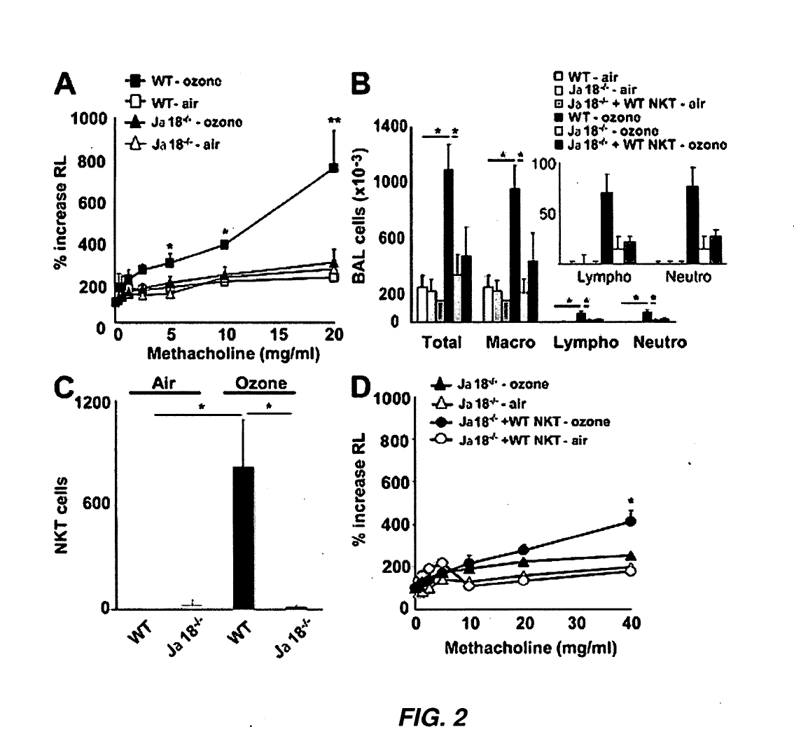

The present invention is based on the discovery that ozone exposure leads to ozone-induced asthma and AHR that are associated with IL-17 production by pulmonary iNKT and T cells. The present invention has shown that that neutralization of IL-17 using anti-IL-17 antibodies prevented the development of ozone-induced but not allergen-induced AHR. While iNKT cells are required for both allergen-induced (13) as well as ozone-induced AHR (FIG. 1A, FIG. 2A), the pulmonary inflammation induced by ozone exposure is very distinct from that induced by allergen. The ozone-induced AHR was characterized by the presence of neutrophils, IL-17, and small number of cells expressing IL-4, whereas the allergen-induced form of AHR was characterized by the presence of eosinophils and with high levels of IL-4 and IL-13 (13). It is believed that the production of IL-17 by pulmonary iNKT and T cells functions indirectly to recruit neutrophils by chemotaxis.

Accordingly, embodied in the present invention is a method of treating non-allergic asthma in an individual. The method includes selecting an individual diagnosed with non-allergic asthma and administering to the individual an effective amount of IL-17 antagonist. In one embodiment, the diagnosis of non-allergic asthma in an individual is made by measuring the level of neutrophils in a sample of sputum described herein. In one embodiment, the non-allergic asthma is ozone-induced asthma.

As used herein, the term “treatment” refers to reducing, ameliorating, stopping, abating, alleviating, and inhibiting the symptoms associated with asthma, such as, respiratory inflammation leading to airway narrowing that causes symptoms such as wheezing, shortness of breath, chest tightness, and coughing. “Treatment” also refers to controlling airway inflammation in order to reduce the reactivity of the airways and also to prevent airway remodeling such as permanent thickening of the bronchial walls as a result of chronic inflammation that does not resolve itself. “Treatment” also include prophylactically preventing the onset of asthmatic symptoms in individual diagnosed with asthma and AHR.

A prophylactically or therapeutically effective amount means that amount necessary to attain, at least partly, the desired effect, of reducing, ameliorating, stopping, abating, alleviating, and inhibiting the symptoms associated with asthma, and also controlling airway inflammation and prevent airway remodeling. Such amounts will depend, of course, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art; however, that a lower dose or tolerable dose can be administered for medical reasons, psychological reasons or for virtually any other reason.

The term “effective amount” refers to an amount that is sufficient to effect a therapeutically or prophylactically significant reduction in asthmatic symptoms and AHR. A therapeutically or prophylactically significant reduction, e.g., frequency and severity of asthma attacks and symptoms in an individual diagnosed as suffering from asthma, is about 10%, about 20%, about 30%, about 40%, about 50%, about 60%, about 70%, about 80%, about 90%, about 100%, or more compared to an individual who does not have suffer from asthma.

Asthma is a chronic disease of the respiratory system in which the airway occasionally constricts, becomes inflamed, and is lined with excessive amounts of mucus, often in response to one or more triggers. Asthma can be defined simply as reversible airway obstruction. These episodes can be triggered by such things as exposure to an environmental stimulant (or allergen), cold air, warm air, moist air, exercise or exertion, or emotional stress. In children, the most common triggers are viral illnesses such as those that cause the common cold (Zhao J., et. al., 2002, J. Pediatr. Allergy Immunol. 13: 47-50). This airway narrowing causes symptoms such as wheezing, shortness of breath, chest tightness, and coughing. The airway constriction responds to bronchodilators. Between episodes, most patients feel well but can have mild symptoms and they can remain short of breath after exercise for longer periods of time than the unaffected individual. The symptoms of asthma, which can range from mild to life threatening, can usually be controlled with a combination of drugs and environmental changes.

The basic diagnosis and measurement of asthma is peak flow rates and the following diagnostic criteria are used by the British Thoracic Society (Pinnock H., and Shah R., 2007, Br. Med. J. 334 (7598): 847-50): ≧20% difference on at least three days in a week for at least two weeks; ≧20% improvement of peak flow following treatment, for example: 10 minutes of inhaled β-agonist (e.g., salbutamol), six week of inhaled corticosteroid (e.g., beclometasone), and 14 days of 30 mg prednisolone; and ≧20% decrease in peak flow following exposure to a trigger (e.g., exercise).

Asthma symptoms can be activated or aggravated by many agents. Not all asthmatics react to the same triggers. Additionally, the effect that each trigger has on the lungs varies from one individual to another. In general, the severity of one's asthma depends on how many agents activate one's symptoms and one's AHR to them. Most of these triggers can also worsen nasal or eye symptoms.

Triggers fall into two categories: allergens (“specific”) and non-allergens—mostly irritants (nonspecific). Once the bronchial tubes (nose and eyes) become inflamed from an allergic exposure, a re-exposure to the offending allergens will often activate symptoms. These “reactive” bronchial tubes could also respond to other triggers, such as exercise, infections, and other irritants. In other words, they have become hyperreactive.

Common allergens that trigger the allergic asthma include “seasonal” pollens, year-round dust mites, molds, pets, and insect parts, foods, such as fish, egg, peanuts, nuts, cow's milk, and soy, additives, such as sulfites, work-related agents, such as latex. Approximately 80% of children and 50% of adults with asthma also have allergies.

Common irritants that can trigger asthma in airways that are hyperreactive include respiratory infections, such as those caused by viral “colds,” bronchitis, and sinusitis, medication drugs, such as aspirin, other NSAIDs (nonsteroidal antiinflammatory drugs), and beta blockers (used to treat blood pressure and other heart conditions), tobacco smoke, outdoor factors such as ozone, smog, weather changes, and diesel fumes; indoor factors such as paint, detergents, deodorants, chemicals, and perfumes; nighttime GERD (gastroesophageal reflux disorder); exercise, especially under cold dry conditions; work-related factors such as chemicals, dusts, gases, and metals; emotional factors, such as laughing, crying, yelling, and distress; and hormonal factors, such as in premenstrual syndrome.

Ozone (O3) is a triatomic molecule, consisting of three oxygen atoms. It is an allotrope of oxygen that is much less stable than the diatomic species O₂. It is thus a powerful oxidizing agent. It is also unstable at high concentrations, decaying to ordinary diatomic oxygen (in about half an hour in atmospheric conditions (www.gcmd.gsfc.nasa.gov/Resources/FAQs/ozone.html). Ground-level ozone is regarded as an air pollutant with harmful effects on the respiratory systems of animals by the World Health Organization (WHO-Europe reports: Health Aspects of Air Pollution, 2003). It is not emitted directly by car engines or by industrial operations. Rather it is formed by the reaction of sunlight on air containing hydrocarbons and nitrogen oxides, pollutants produced by industrial operations, that react to form ozone directly at the source of the pollution or many kilometers down wind. It is believed that the powerful oxidizing properties of ozone can be a contributing factor of inflammation. Asthma related to ozone exposure is characterized by neutrophilia airway inflammation (Mehrdad Arjomandi, et. al., Chest, 2005; 128:416-423).

In one embodiment, the diagnosis of non-allergic asthma in an individual is made by measuring the level of neutrophils in a sample of sputum. Sputum induction can be performed, for example, by inhalation of nebulized saline at increasing concentrations (up to 5%) according to a method described by Pizzichini, E., et. al., Am. J. Respir. Crit. Care Med. 1996; 154, 308-317. Briefly, the individual receives two puffs of salbutamol (100 μg/puff) 15 min prior to the procedure, and then inhales 4% hypertonic saline solution delivered by an ultrasonic nebulizer device (Ultra Neb 2000; DeVilbiss; Wolleston, UK) for 15 min. The individual then rinse his or her mouth, blow his or her nose, and carefully cough sputum into a Petri dish using forced expiratory maneuvers. The first portion of sputum is discarded, and the inhalation procedure is continued for a further 15 min. Lung function is carefully monitored by spirometry every 5 min during induction to ascertain safety of the procedure. Induction is stopped when the total length of induction of 15 min is completed or a >15% drop in FEV1 occurred. In the latter scenario, the individual can be administered two additional puffs of salbutamol. Mucus plugs are manually removed and fixed immediately in 4% paraformaldehyde in PBS for 2 h. Samples were paraffin embedded via butanol in a tissue array fashion (Musat-Marcu, S., et. al., J. Mol. Cell. Cardiol., 1999, 31:1073). Cells can be counterstained with 4′,6′-diamidino-2-phenylindole nuclear stain.

In another embodiment, sputum and saliva samples can be added to an equal volume of a 0.1% dithiothreitol-saline solution (Sigma, St. Louis, Mo.). They are gently mixed with a vortex mixer and placed in a water bath at 37° C. for 15 min, with periodic removal from the water bath for further brief gentle mixing. Homogenized samples is then centrifuged at 800 g for 10 min, and the supernatants are aspirated.

The cell pellets are resuspended in saline, and the total cell count and viability are assessed in 10-μl aliquots with a standard hemacytometer and Trypan blue exclusion, respectively. The cells can then be cytocentrifuged (Cytospin 2, Shandon Instruments, Runcorn, UK) and stained with Diff-Quik (Merz-Dade, Dudingen, Switzerland) or Leishman's, Giemsa, Jenner's, or Wright's stain. The slides should be blindly read by two investigators who will count at least 400 cells/slide. The squamous cell count was subtracted from the total cell count, and the differential counts are expressed as corrected percentages. Sputum samples were considered adequate if the sputum volume was at least 1 ml with <50% squamous cells on differential count. Polymorphonuclear neutrophils (PMN) counting can be performed according to methods known in the art and described in A. M. Li, et. al., Thorax 2006; 61:747-750. PMN are identified based on their multi-lobular nuclear morphology and light general staining compared to eosinophils, which stain well with eosin and contain numerous cytolpasmic small granulues.

In a healthy individual, the total cell count (TCC) in a sputum sample is less than 4.5 million cells per milliliter (ml), with less than 37% are neutrophils and less than 2% are eosinophils. TCC does not include squamous epithelial cells. Accordingly, an individual is considered to have elevated neutrophils in the sputum if the percentage of neutrophils in the sputum is more than 37% of the TCC, about 40%, about 50%, about 60%, about 70%, about 80%, about 90%, about 99%, and all the percentages in between 37% and 100%, of the TCC/ml. The individual can also have elevated TCC, at least greater than 4.5×10⁶ cells/ml.

In one embodiment, the methods described herein involved administering an IL-17 antagonist to an individual with elevated neutrophils in the sputum. As used herein, the term “an IL-17 antagonist” refers to any organic, inorganic molecule, or biologic that can blocks, inhibits, and prevents the cytokine IL-17A ligand-to-IL-17 receptor interaction and the consequent downstream signaling pathway. An IL-17A antagonist can also reduce the effective amount of endogenous biologically active IL-17 available for binding to the receptor or it can prevent the binding of IL-17 to its receptor. Such antagonists include receptor-binding peptide fragments of IL-17, antibodies directed against IL-17 (antibodies that bind IL-17 and inhibit binding thereof to IL-17 receptor), antibodies directed against IL-17 receptor (antibodies that bind IL-17 receptor and inhibit receptor binding of IL-17 without themselves transducing a signal via IL-17 receptor), soluble forms of IL-17 receptor, molecules that bind IL-17 or IL-17 receptor and inhibit the interaction thereof and polypeptides comprising all or portions of receptors for IL-17 or modified variants thereof, including genetically-modified muteins, multimeric forms and sustained-release formulations thereof. Particular antagonists are soluble forms of IL-17 receptor. Other particular IL-17 antagonists encompass chimeric proteins that include portions of both an antibody molecule and an IL-17 antagonist molecule, particularly a soluble portion of IL-17 receptor fused to an Fc. Such chimeric molecules can form monomers, dimers or higher order multimers. Preferred methods of the invention utilize IL-17 receptor in a form that binds IL-17 and blocks IL-17 signal transduction, thereby interrupting the proinflammatory and immunoregulatory effects of IL-17, for example, soluble IL-17 receptor. Other antagonist can be nucleic acids molecules that reduces the expressions of the endogenous genes that encode IL-17A and IL-17A receptor, such as an antisense IL-17 nucleic acid molecule, an antisense IL-17 receptor nucleic acid molecule, an siRNA IL-17 nucleic acid molecule, and an siRNA IL-17 receptor nucleic acid molecule.

As used herein, the term “nucleic acid” refers to high-molecular-weight nucleotide polymer such as deoxyribonucleic acid (DNA) and ribonucleic acid (RNA).

As used herein, the term “endgogenous gene” refers to the original copy of gene found in the genome of the individual.

As used herein, the term “comprising” means that other elements can also be present in addition to the defined elements presented. The use of “comprising” indicates inclusion rather than limitation.

In one embodiment, a method of treating an individual at risk of non-allergic asthma is provided. The method comprises measuring the level of neutrophils in a sample of sputum from an individual and administering to the individual an effective amount of an IL-17 antagonist when the level of neutrophils is above a predetermined level. The individual is considered at risk of non-allergic asthma if the individual has a previous non-allergic asthma attack and if the individual has an elevated percentage of neutrophils in the lung sputum as determined by the methods described herein.

In one embodiment, the methods described herein further comprise administering an effective amount of NKT cell inhibitory agent. The NKT cell inhibitory agent inhibits NKT cell function through the interaction of the NKT cell with an antigen presenting molecule. In the experimental model of ozone-induced asthma described in the example, both NKT cells and T cells in the lungs produced IL-17A that lead to recruitment of neutrophils. The treatment of ozone-induced asthma with a combination of inhibiting NKT cells and IL-17 antagonist can be more effective than treatment with just an IL-17 antagonist since inhibiting NKT cells serves to reduce the overall IL-17 production. Examples of NKT cell inhibitory agent are glycolipds such as glucocerebrosides (Maya Margalit, et. al., Am J Physiol Gastrointest Liver Physiol 289: G917-G925, 2005) and β-galactosylceramide (US Patent Application No. 2006/0116332), and IL-6 (Rui Sun, et. al., J Immunol., 2004, 172: 5648-5655). These references are hereby incorporated by reference in their entirety.

In another embodiment, the methods described herein for treating ozone induced asthma comprise administering a combination of IL-17 antagonists. For example, the combination can be a soluble IL-17R and an siRNA IL-17A nucleic acid, a soluble IL-17R and an antisense IL-17 nucleic acid, and an inhibitory anti-IL-17 antibody and a fusion protein comprising IL-17R. All possible combinations of a soluble form of an IL-17 receptor, an inhibitory anti-IL-17 antibody, an inhibitory anti-IL-17R antibody, a fusion protein comprising IL-17 receptor, a fusion protein comprising IL-17 binding fragment of IL-17 receptor, an IL-17 antagonistic small molecule, an antisense IL-17 nucleic acid molecule, an antisense IL-17 receptor nucleic acid molecule, an siRNA IL-17 nucleic acid molecule, and an siRNA IL-17 receptor nucleic acid molecule are envisioned in the methods described herein. In another embodiment, the methods for treating ozone induced asthma comprise administering an NKT cell inhibitory agent and a combination of IL-17 antagonists.

Unless otherwise defined herein, scientific and technical terms used in connection with the present application shall have the meanings that are commonly understood by those of ordinary skill in the art to which this disclosure belongs. Definitions of common terms immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Further, unless otherwise required by context, singular terms shall include pluralities and plural terms shall include the singular.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless otherwise stated, the present invention was performed using standard procedures known in the art, and as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

Soluble or Fusion IL-17R

interleukin 17A (IL-17A) is a pro-inflammatory cytokine secreted by activated T-lymphocytes. IL-17A is also known as CTLA-8, as it was first identified as a cDNA cloned from an activated T cell hybridoma clone (Rouvier et al., J. Immunol. 150:5445; 1993). It is a potent inducer of the maturation of CD34-positive hematopoietic precursors into neutrophils.

This pro-inflammatory cytokine IL-17A acts on cells by binding to a specific receptor, IL-17R, which was identified by Yao Z., et. al, Cytokine 9: 794-800 (1997) and isolated as described U.S. Pat. No. 6,072,033, which are incorporated herein by reference in its entirety. The IL-17R is a ubiquitous type I membrane glycoprotein that binds with low affinity to interleukin 17A. The IL-17R has an N-terminal signal peptide with a predicted cleavage site between amino acid 27 and 28. The signal peptide is followed by a 293 amino acid extracellular domain, a 21 amino acid transmembrane domain, and a 525 amino acid cytoplasmic tail. Soluble forms of the receptor can be prepared and used to sequester the cytokine IL-17A and regulate immune responses in a therapeutic setting. Accordingly, pharmaceutical compositions comprising soluble forms of IL-17R are useful for the methods described herein. Deleted forms and fusion IL-17R proteins comprising the IL-17A binding regions, and homologs thereof are also disclosed.

The nucleic acid of human IL-17R can be found in Genbank Accession No. NM_{—}014339 (SEQ. ID. No. 1) and the amino acid sequence of the human IL-17R is found in Genbank Accession No. NP_{—}055154 and AAB99730 (SEQ. ID. No. 2). Soluble forms of IL-17R that are useful in the methods described herein include the extracellular domain (residues 1-320 of SEQ. ID. No. 2 or residues 28-320 which excludes the signal peptide) or a fragment of the extracellular domain that has the properties of antagonizing or preventing binding of IL-17 receptor to IL-17. Variants of soluble forms of IL-17R having conserved amino acid substitution are also envisioned herein. Other forms of IL-17R that are useful in the methods described herein include muteins and conservatively modified variations that are at least 70% identical to the native IL-17 receptor protein (SEQ. ID. No. 2) or at least 90% identical to the coding nucleic acid of the native IL-17 receptor (SEQ ID No: 1) and as described in U.S. Pat. No. 6,072,033, which is incorporated herein by reference in its entirety. A particularly preferred form of a IL-17 inhibitor is a soluble IL-17R, which is described in detail in U.S. Pat. No. 5,869,286, which is incorporated herein by reference in its entirety.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ea., Oxford University Press, New York, 1988; Biocomputing: Informatics and −14 Genome Projects, Smith, D. W., ea., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988)). Methods to determine identity are designed to give the largest match between the sequences tested. Moreover, methods to determine identity are codified in publicly available computer programs such as BLASTP.

As used herein, the term “conservative amino acid substitution” is one in which the amino acid residue is replaced with an amino acid residue having a side chain with a similar charge and size. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins, 1984).

“Conservatively modified variations” or “variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

It is envisioned that fusion proteins of soluble IL-17R containing a fragment of the extracellular domain that has the properties of antagonizing or preventing binding of IL-17 receptor to IL-17 are also useful for the methods described herein. Fusion proteins of soluble IL-17R can be synthesize as recombinant C-terminus or N-terminus fusion proteins with (1) tags such as His-tag (6x-histidine-tag) (SEQ. ID. No. 5) to facilitate purification and identification of the fusion protein; (2) an immunoglobulin Fc region, the Fc domains having reduced affinity for Fc receptors are preferred for the purification of Fc fusion protein and dimerization of Fc-fusion protein, and the Fc domain aid in increasing the serum half-life of the Fc-fusion protein; and (3) an oligomerization zipper domains for dimerization of the soluble IL-17R, such as a leucine zipper domain.

The ability of various soluble IL-17R and fusion IL-17R proteins to bind IL-17A (CTLA-8) can be determined by testing the ability of the IL-17R proteins to inhibit IL-17A (CTLA-8)-induced T cell proliferation. Alternatively, suitable assays, for example, an enzyme immunoassay or a dot blot, employing CTLA-8 or HSV13 (or a homolog thereof which binds native IL-17R) can be used to assess the ability of IL-17R analogs to bind IL-17A. Such methods are well known in the art.

The cloning, expression, and purifications of soluble IL-17R and soluble fusion IL-17R are performed by methods know in the art and according to methods described in U.S. Pat. Nos. 5,869,286, 6,072,033, 6,072,037, 6,083,906, 6,096,305, 6,100,235, 6,191,104, 6,680,057, 6,706,870, and 6,793,919, and these are incorporated herein by reference in their entirety.

Inhibitory Antibodies

In one embodiment, IL-17 antagonists are inhibitory antibodies against cytokine IL-17A and IL-17R. Such inhibitory antibodies can act by sequestering the cytokine IL-17A, sterically hindering IL-17A interacting with IL-17R, and clustering of IL-17R. Commercially available antibodies including mouse IgG monoclonal antibody against the human IL-17A (Anti-IL-17), catalog number: 20-003-40008, can be obtained from GenWay BioTech Inc. San Diego, Calif., Santa Cruz Biotechnology, Inc. USA, and Immunex (now Amgen) Inc., and Mouse IgG2B monoclonal anti-human IL-17R antibody catalog number: MAB1771 can be obtained from R & D systems, Minneapolis, Minn. USA. Alternatively, antibodies can be generated and synthesized by methods known in the art.

The antibodies can be polyclonal or monoclonal antibodies. Antibodies are raised against the human IL-17A protein (SEQ. ID. No. 3; Genbank Accession No.: NP_{—}002181.1, AAH67505, AAH67503, AAH67504, AAH66253, AAH66251) or the human IL-17R Genbank Accession No. NP_{—}055154 and AAB99730 (SEQ. ID. No. 2). Alternatively, antibodies can be made by immunizing a mammal with an inoculum containing a recombinant DNA molecule that comprises a DNA sequence that contains a sequence encoding the human IL-17A or IL-17R. The recombinant DNA sequences are derived from the human IL-17A nucleic acid (SEQ. ID. No. 4; Genbank Accession No.: NM_{—}002190, AL391221, BC 067505, BC067503, BC067504, BC066253, BC066251, BC066252) and the nucleic acid of human IL-17R Genbank Accession No. NM_{—}014339 (SEQ. ID. No. 1). Such method is described in U.S. Pat. No. 5,643,578 which is incorporated herein by reference in its entirety.

Methods for the production of antibodies against IL-17R are described in U.S. Pat. Nos. 6,072,037, 6,793,919, and WO 2007/070750 which are herein incorporated by reference in their entirety. Inhibitory antibodies envisioned for the methods described herein include humanized antibodies, chimeric antibodies (e.g. an antibody with mouse variable region fused with human constant region), single chain antibodies, single-domain antibody, variant forms of humanized, chimeric or single chain antibodies that conserved amino acid substitutions at the non-antigen binding region such as in the immunoglobulin constant region (Fc), and any protein containing the antigen binding region of any inhibitory IL-17A or IL-17R antibody, including the Fab, F(ab)′2 or Fv fragment.

The inhibitory effect of the antibodies on IL-17 signally pathway can be determined by testing the ability of the antibodies to inhibit IL-17 (CTLA-8)-induced T cell proliferation. Such methods are well known in the art.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

The design of humanized immunoglobulins can be carried out as follows. When an amino acid falls under the following category, the framework amino acid of a human immunoglobulin to be used (acceptor immunoglobulin) is replaced by a framework amino acid from a CDR-providing non-human immunoglobulin (donor immunoglobulin): (a) the amino acid in the human framework region of the acceptor immunoglobulin is unusual for human immunoglobulins at that position, whereas the corresponding amino acid in the donor immunoglobulin is typical for human immunoglobulins in that position; (b) the position of the amino acid is immediately adjacent to one of the CDRs; or (c) the amino acid is capable of interacting with the CDRs (see, Queen et al. WO 92/11018., and Co et al., Proc. Natl. Acad. Sci. USA 88, 2869 (1991), respectively, both of which are incorporated herein by reference). For a detailed description of the production of humanized immunoglobulins see, Queen et al. and Co et. al.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies.

The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (Skerra et al., Science 242:1038-1041 (1988)).

Methods for the production of antibodies are disclosed in PCT publication WO 97/40072 or U.S. Application. No. 2002/0182702, which are herein incorporated by reference. The processes of immunization to elicit antibody production in a mammal, the generation of hybridomas to produce monoclonal antibodies, and the purification of antibodies may be performed by described in “Current Protocols in Immunology” (CPI) (John Wiley and Sons, Inc.) and Antibodies: A Laboratory Manual (Ed Harlow and David Lane editors, Cold Spring Harbor Laboratory Press 1988) which are both incorporated by reference herein in their entirety.

Once expressed, the whole antibodies, their dimers, individual light and heavy chains, or other immunoglobulin forms of the present invention can be purified according to standard procedures in the art, including ammonium sulfate precipitation, affinity columns, column chromatography, gel electrophoresis and the likes (see, generally, Scopes, R., Protein Purification, Springer-Verlag, N.Y. (1982), which is incorporated herein by reference in its entirety). Substantially pure immunoglobulins of at least about 90 to 95% homogeneity are preferred, and 98 to 99% or more homogeneity most preferred, for pharmaceutical uses.

Small Molecule IL-17 Antagonist

In one embodiment, pharmacological agents or lead compounds for agents that have properties of antagonizing IL-17R and/or preventing binding of IL-17R to IL-17A are also useful for the methods described herein. Screening assays for agonists and antagonists of receptor-ligand interactions as well known in the art (see U.S. Pat. No. 6,884,598, which is hereby incorporated by reference in its entirety). For example, testing the ability of an agent or compound to inhibit IL-17A-induced T cell proliferation. In the presence of an agent or compound, a reduction of IL-17A induced T cell proliferation by at least 10%, 20%, 30%, 40%, 50%, 60%, 70%, 80%, 90%, 100% and all the percentages between 10% and 100% compared to the IL-17A induced T cell proliferation in the absence of any added agent or compound is a positive indication of IL-17 antagonist activity in that agent or compound. Alternatively, an agent or compound can be tested by an enzyme immunoassay or a dot blot for its ability to inhibit IL-17R-IL-17A interaction. In the presence of an agent or compound, a reduction of IL-17A bound to IL-17R by least 10%, 20%, 30%, 40%, 50%, 60%, 70%, 80%, 90%, 100% and all the percentages between 10% and 100% compared to the amount of IL-17A bound to IL-17R in the absence of any added agent or compound indicates that the agent or compound has IL-17A antagonist activity. In one embodiment, the identification of test compounds capable of modulating IL-17A activity is performed using high-throughput screening assays, such as BIACORE® (Biacore International AB, Uppsala, Sweden), BRET (bioluminescence resonance energy transfer), and FRET (fluorescence resonance energy transfer) assays, as well as ELISA and cell-based assays.

Decreased IL-17A activity in an individual afflicted with non-allergen induced asthma can also be achieved through the use of small molecules (usually organic small molecules) that antagonize, i.e., inhibit the activity of, IL-17A. Novel antagonistic small molecules can be identified by the screening methods described herein and can be used in the treatment methods described herein.

The term small molecule refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2nd ed., ed. N. Rose, Wiley and Sons, New York, 14 (1995)). For example, Davis et al. (2005) Proc. Natl. Acad. Sci. USA 102:5981-86, use the phrase small molecule to indicate folates, methotrexate, and neuropeptides, while Halpin and Harbury (2004) PLos Biology 2:1022-30, use the phrase to indicate small molecule gene products, e.g., DNAs, RNAs and peptides. Examples of natural small molecules include, but are not limited to, cholesterols, neurotransmitters, aptamers and siRNAs; synthesized small molecules include, but are not limited to, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24).

Inhibition of TL-17A and TL-17R Expression

In one embodiment, antisense nucleic acid technology and RNA interference are used as therapies for treating non-allergen induced asthma. Antisense therapy is a form of treatment where when the genetic sequence of a particular gene is known to be causative of a particular disease. The goal is the reduction or elimination of the expression of that particular gene. It is possible to synthesize a strand of nucleic acid (DNA, RNA or a chemical analogue) that will bind to the messenger RNA (mRNA) produced by that gene and inactivate it, effectively turning that gene “off”. This is because mRNA has to be single stranded for the related protein to be translated. This synthesized nucleic acid is termed an “anti-sense” oligonucleotide because its base sequence is complementary (i.e. can complementary base-pair) to the gene's messenger RNA (mRNA), which is called the “sense” sequence.

As used herein, the term “complementary base pair” refers to A:T and G:C in DNA and A:U in RNA. Most DNA consists of sequences of nucleotide only four nitrogenous bases: base or base adenine (A), thymine (T), guanine (G), and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

In one embodiment, the methods disclosed herein comprise the use of an inhibitory oligonucleotide or combinations of inhibitory oligonucleotides, or combinations of inhibitory oligonucleotides and IL-17 antagonist to reduce the effects of Il-17A-IL-17R interaction. Antisense nucleic acid technology and RNA interference both comprise the use of inhibitory oligonucleotide such as siRNA, microRNA, and anti-sense oligonucleotides.

RNA interference (RNAi) is a phenomenon in which double-stranded RNA (dsRNA) specifically suppresses the expression of a gene with its complementary sequence. Small interfering ds RNAs (siRNA) mediate post-transcriptional gene-silencing, and can be used to induce RNAi in mammalian cells.

Short antisense nucleic acid sequences complementary to the coding regions of IL-17A and IL-17R (SEQ. ID. Nos. 4 and 2) can be chemically synthesized and enzymatically ligated by methods known in the art. Alternately, the antisense oligonucleotide has complementary sequences to the noncoding regions of IL-17A and IL-17R, such as the genomic sequence of IL-17A and IL-17R. The preferably length of a typical antisense nucleic acid or siRNA is between 10-30 bases. This length is optimal for cell entry without involving carrier systems and for effective targeting to mRNA of interest. Special chemical modifications such as sulfonation to give rise to a phosphorothioate backbone of the oligonucleotide and by substituting all 2′-OH residues on the RNA with 2′F, 2′O-Me or 2′H residues dramatically improves the in vivo stability of siRNA and antisense oligonucleotides by helping the nucleic acids elude immediate nuclease degradation while maintaining the ability to attract Rnase H for mRNA-antisense degradation. Methods of predicting and selecting antisense oligonucleotides and siRNA are known in the art and are also found at www.genscript.com/ssl-bin/app/rnai, www.ambion.com/techlib/misc/siRNA_finder.html, www.dharmacon.com/DesignCenter, www.oligoengine.com, http://sfold.wadsworth.org/sirna.pl, www.genscript.com/ssl-bin/app/rnai, http://jura.wi.mit.edu/bioc/siRNAext/home.php and described in U.S. Pat. No. 6,060,248 which is hereby incorporated by reference in its entirety. Alternatively, these molecules can be produced biologically using an expression vector carrying the nucleic acid that encode the siRNA or antisense RNA.

The inhibitory antisense and siRNAs described herein also include triplex-forming oligonucleotides (TFOs) that bind in the major groove of duplex DNA with high specificity and affinity (Knauert and Glazer (2001) Hum. Mol. Genet. 10:2243-51). Expression of IL-17A and IL-17R genes related can be inhibited by targeting TFOs complementary to the regulatory regions of the genes (i.e., the promoter and/or enhancer sequences) to form triple helical structures that prevent transcription of the genes.

In one embodiment of the invention, the inhibitory oligonucleotides are short interfering ds RNA (siRNA). These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (see, e.g., Bass, 2001, Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nature Med. 9:347-51). In addition, intrathecally delivered siRNA has recently been reported to block pain responses in two models (agonist-induced pain model and neuropathic pain model) in the rat (Dom et al. (2004) Nucleic Acids Res. 32(5):e49).

The siRNA molecules of the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express short hairpin RNAs (shRNA), which are further processed into siRNAs (Arts et al. (2003) Genome Res. 13:2325-32). These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.). Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

Reducing the expression of the LI-17A and IL-17R genes in an organism can also be achieved through the creation of an expression vector that expressed the antisense oligonucleotide. A tissue-specific regulatory sequence(s) can be operably linked to the antisense sequence to direct transcription in only the appropriate tissue. Methods for generating are well known in the art (e.g., Xing H., et. al., Zhonghua Shi Yan He Lin Chuang Bing Du Xue Za Zhi. 2002 16:52-4; Elke I. Behrend, et. al., in Annals of the New York Academy of Sciences, 1995, 760: 299-301; and U.S. Pat. No. 6,943,022).

As used herein, “reducing expression” or “gene silencing” includes any decrease in expression or protein activity or level of the target gene or protein encoded by the target gene as compared to a situation wherein no RNA interference has been induced. The decrease can be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

The siRNA molecules targeted to the polynucleotides related to the present invention can be designed based on criteria well known in the art (e.g., Elbashir et al. (2001) EMBO J. 20:6877-88). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target mRNA and preferably should be at least 75 by after the initiation ATG and at least 75 by before the stop codon. Based on these criteria, or on other known criteria (e.g., Reynolds et al. (2004) Nature Biotechnol. 22:326-30), siRNA molecules related to the present invention that target the mRNA polynucleotides related to the present invention can be designed by one of ordinary skill in the art.

The targeted region of the siRNA molecule of the present invention can be selected from a given target gene sequence, e.g., the IL-17A or IL17R coding sequence, beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a siRNA molecule of the present invention involves identifying the 23 nucleotide sequence motif AA(N19)TT (SEQ. ID. No. 6) (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but are not limited to the NCBI, BLAST, Derwent and GenSeq as well as commercially available oligosynthesis software such as Oligoengine®, can also be used to select siRNA sequences against EST libraries to ensure that only one gene is targeted.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotidesmolecules similar to the oligonucleotides containing 2′-O-methyl ribose, arabinose and particularly D-arabinose (U.S. Pat. No. 5,177,196).

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

In some embodiments, the inhibitory oligonucleotides are short-hairpin RNA (shRNA), small temporal RNA (stRNA), microRNA (miRNA), RNA mimetics, or heterochromatic siRNA. In other embodiments, the inhibitory oligonucleotides are but not limited to DNA, RNA, peptide-nucleic acid (PNA), pseudo-complementary PNA (pc-PNA), or locked nucleic acid (LNA), phosphorodiamidate morpholino oligomer and the like.

Locked nucleic acids (LNAs), also known as bridged nucleic acids (BNAs), developed by Wengel and co-workers (Koshkin A. A., 1998, Tetrahedron, 54:3607-3630) and Imanishi and co-workers (Obika S., 1998, Tetrahedron Lett., 39:5401-5404). LNA bases are ribonucleotide analogs containing a methylene linkage between the 2′ oxygen and the 4′ carbon of the ribose ring. The constraint on the sugar moiety results in a locked 3′-endo conformation that preorganizes the base for hybridization and increases melting temperature (Tm) values as much as 10° C. per base (Wengel J., 1999, Acc. Chem. Res., 32:301-310; Braasch D. A. and Corey, D. R., 2001, Chem. Biol., 8:1-7). LNA bases can be incorporated into oligonucleotides using standard protocols for DNA synthesis. This commonality facilitates the rapid synthesis of chimeric oligonucleotides that contain both DNA and LNA bases and allows chimeric oligomers to be tailored for their binding affinity and ability to activate RNase H. Because oligomers that contain LNA bases have a native phosphate backbone they are readily soluble in water. Introduction of LNA bases also confers resistance to nucleases when incorporated at the 5′ and 3′ ends of oligomers (Crinelli R., et. al., 2002, Nucleic Acids Res., 30:2435-2443). The ability to use LNAs for in vivo applications is also favored by the finding that LNAs have demonstrated low toxicity when delivered intravenously to animals (Wahlestedt C., et. al., 2000, Proc. Natl. Acad. Sci. USA, 97: 5633-5638).

LNAs and LNA-DNA chimeras have been shown to be potent inhibitors of human telomerase and that a relatively short eight base LNA is a 1000-fold more potent agent than an analogous peptide nucleic acid (PNA) oligomer (Elayadi A. N., et. al., 2002, Biochemistry, 41: 9973-9981). LNAs and LNA-DNA chimeras have also been shown to be useful agents for antisense gene inhibition. Wengel and co-workers have used LNAs to inhibit gene expression in mice (Wahlestedt C., et. al., 2000, Proc. Natl. Acad. Sci. USA, 97:5633-5638), while Erdmann and colleagues have described the design of LNA-containing oligomers that recruit RNase H and have described the rules governing RNase H activation by LNA-DNA chimeras in cell-free systems (Kurreck J., et. al., 2002, Nucleic Acids Res., 30:1911-1918).

The syntheses of LNA-containing oligomers are known in the art, for examples, those described in U.S. Pat. No. 6,316,198, 6,670,461, 6,794,499, 6,977,295, 6,998,484, 7,053,195, and U.S. Patent Publication No. US 2004/0014959, and all of which are hereby incorporated by reference in their entirety.

Phosphorodiamidate morpholino oligomers (PMOs) are DNA mimics that inhibit expression of specific mRNA in eukaryotic cells (Arora, V., et. al., 2000, J. Pharmacol. Exp. Ther. 292:921-928; Qin, G., et. al., 2000, Antisense Nucleic Acid Drug Dev. 10:11-16; Summerton, J., et. al., 1997, Antisense Nucleic Acid Drug Dev. 7:63-70). They are synthesized by using the four natural bases, with a base sequence that is complementary (antisense) to a region of a specific mRNA. They are different than DNA in the chemical structure that links the bases together. Ribose has been replaced with a morpholine group, and the phosphodiester is replaced with a phosphorodiamidate. These alterations make the antisense molecule resistant to nucleases (Hudziak, R., et. al., 1996 Antisense Nucleic Acid Drug Dev. 6:267-272) and free of charges at physiological pH, yet it retains the molecular architecture required for binding specifically to a complementary strand of nucleic acid (Stein, D., et. al, 1997, Antisense Nucleic Acid Drug Dev. 7:151-157; Summerton, J., et. al., 1997, Antisense Nucleic Acid Drug Dev. 7:63-70; Summerton, J., and D. Weller., 1997, Antisense Nucleic Acid Drug Dev. 7:187-195).

The synthesis, structures, and binding characteristics of morpholine oligomers are detailed in U.S. Pat. Nos. 5,698,685, 5,127,866, 5,142,047, 5,166,315, 5,521,063, and 5,506,337, and all of which are hereby hereby incorporated by reference in their entirety. Phosphorodiamidate morpholino oligomers (PMOs) can be synthesized at AVI BioPharma (Corvallis, Oreg.) in accordance with known methods, as described, for example, in Summerton, J., and D. Weller U.S. Pat. No. 5,185,444; and Summerton, J., and D. Weller. 1997, Antisense Nucleic Acid Drug Dev. 7:187-195. Methods of making and using PMO for the inhibition of gene expression in vivo are described in U.S. Patent Publication No. US 2003/0171335; US 2003/0224055; US 2005/0261249; US 2006/0148747; S 2007/0274957; US 2007/003776; and US 2007/0129323; and these are hereby incorporated by reference in their entirety.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

In a preferred embodiment, the siRNA or modified siRNA is delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting IL-17A or IL-17R. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Bio sciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

Examples of some commercial vectors for the production and delivery of short hairpin RNA (shRNA) include but are not limited to #AM5779:-pSilencer™ 4.1-CMV neo; #AM5777:-pSilencer™ 4.1-CMV hygro; #AM5775:-pSilencer™ 4.1-CMV puro; #AM7209:-pSilencer™ 2.0-U6; #AM7210:-pSilencer™ 3.0-H1; #AM5768:-pSilencer™ 3.1-H1 puro; #AM5762:-pSilencer™ 2.1-U6 puro; #AM5770:-pSilencer™ 3.1-H1 neo; #AM5764:-pSilencer™ 2.1-U6 neo; #AM5766:-pSilencer™ 3.1-H1 hygro; #AM5760:-pSilencer™ 2.1-U6 hygro; #AM7207:-pSilencer™ 1.0-U6 (circular) from Ambion®.

Delivery of RNA Interfering Agents: Methods of delivering RNA interfering agents, e.g., an siRNA, or vectors containing an RNA interfering agent, to the target cells (e.g., cells of the brain or other desired target cells, for cells in the central and peripheral nervous systems), can include, for example (i) injection of a composition containing the RNA interfering agent, e.g., an siRNA, or (ii) directly contacting the cell, e.g., a cell of the brain, with a composition comprising an RNA interfering agent, e.g., an siRNA. In one embodiment, the RNA interfering agent can be targeted to the bone marrow where the lymphocytes expressing IL-17 are made. In another embodiment, RNA interfering agents, e.g., an siRNA can be injected directly into any blood vessel, such as vein, artery, venule or arteriole, via, e.g., hydrodynamic injection or catheterization. In yet another embodiment, the RNA interfering agent can be injected or applied topically directly to the site of the skin ulcers.

Administration can be by a single injection or by two or more injections. The RNA interfering agent is delivered in a pharmaceutically acceptable carrier. One or more RNA interfering agents can be used simultaneously. The RNA interfering agents, e.g., the siRNAs targeting IL-17A or IL-17R mRNA, can be delivered singly, or in combination with other RNA interfering agents, e.g., siRNAs, such as, for example siRNAs directed to other cellular genes. IL-17A and/or IL-17R siRNAs can also be administered in combination with other pharmaceutical agents which are used to treat or prevent neurodegenerative diseases or disorders.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501).

RNA interfering agents, for e.g., an siRNA, can also be introduced into cells via the vascular or extravascular circulation, the blood or lymph system, and the cerebrospinal fluid.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

It is also known that RNAi molecules do not have to match perfectly to their target sequence. Preferably, however, the 5′ and middle part of the antisense (guide) strand of the siRNA is perfectly complementary to the target nucleic acid sequence.

Formulation and Composition

In one embodiment, the methods described herein comprise administering a pharmaceutical composition comprising an IL-17 antagonist and a pharmaceutically acceptable carrier.

As used herein, the term “pharmaceutical composition” refers to an IL-17 antagonist or combinations of IL-17 antagonists described herein and a pharmaceutically acceptable carrier of chemicals and compounds commonly used in the pharmaceutical industry. The term “pharmaceutically acceptable carrier” excludes tissue culture medium.

All dosage forms of the pharmaceutical composition, along with methods for their preparation, are well known in the pharmaceutical and cosmetic art (see HARRY'S COSMETICOLOGY (Chemical Publishing, 7th ed. 1982); REMINGTON'S PHARMACEUTICAL SCIENCES (Mack Publishing Co., 18th ed. 1990)). Other desirable ingredients for use in such preparations include preservatives, co-solvents, viscosity building agents, carriers, etc. The carrier itself or a component dissolved in the carrier may have palliative or therapeutic properties of its own, including moisturizing, cleansing, or anti-inflammatory/anti-itching properties. Penetration enhancers may, for example, be surface active agents; certain organic solvents, such as di-methylsulfoxide and other sulfoxides, dimethyl-acetamide and pyrrolidone; certain amides of heterocyclic amines, glycols (e.g. propylene glycol);propylene carbonate; oleic acid; alkyl amines and derivatives; various cationic, anionic, nonionic, and amphoteric surface active agents; and the like.

In one embodiment, dosage forms include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. Nos. 3,773,919, 3,887,699, EP 58,481A, EP 158,277A, Canadian Patent No. 1176565, U. Sidman et al., Biopolymers 22:547 (1983) and R. Langer et al., Chem. Tech. 12:98 (1982). Biologics such as antibodies and proteins will usually be formulated at a concentration of about 0.1 mg/ml to 100 mg/ml and the viral vector that carry the gene for expressing the biologics in vivo should be in the range of 10⁽⁶⁾ to 1×10⁽¹⁴⁾ viral vector particles per application per patient.

In one embodiment, other ingredients can be added to pharmaceutical formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the pharmaceutical formulation to be used for therapeutic administration must be sterile. Sterility is readily accomplished by filtration through sterile filtration membranes (e.g., 0.2 micron membranes). The IL-17 antagonist ordinarily can be stored in lyophilized form or as an aqueous solution if it is highly stable to thermal and oxidative denaturation. The pH of the IL-17 antagonist preparations typically will be about from 6 to 8, although higher or lower pH values may also be appropriate in certain instances.

The pharmaceutical compositions described herein can also be administered systemically in a pharmaceutical formulation. The preferred formulation is also sterile saline or Lactated Ringer's solution. Lactated Ringer's solution is a solution that is isotonic with blood and intended for intravenous administration. Systemic routes include but are limited to oral, parenteral, nasal inhalation, intratracheal, intrathecal, intracranial, and intrarectal. The pharmaceutical formulation is preferably a sterile saline or lactated Ringer's solution. For therapeutic applications, the preparations described herein are administered to a mammal, preferably a human, in a pharmaceutically acceptable dosage form, including those that may be administered to a human intervenously as a bolus or by continuous infusion over a period of time, by intramuscular, intraperitoneal, intracerebrospinal, subcutaneous, intra-arterial, intrasynovial, intrathecal, oral, topical, or inhalation routes. A preferred embodiment is the nasal inhalation of IL-17 antagonist formulated for used in a nebulizer. Viral vectors encoding an IL-17 antagonist can be formulated for use with a nebulizer. For these uses, additional conventional pharmaceutical preparations such as tablets, granules, powders, capsules, and sprays may be preferentially required. In such formulations further conventional additives such as binding-agents, wetting agents, propellants, lubricants, and stabilizers may also be required. In one embodiment, the therapeutic compositions described herein are formulated in a cationic liposome formulation such as those described for intratracheal gene therapy treatment of early lung cancer (Zou Y. et. al., Cancer Gene Ther. 2000 May; 7(5):683-96). The liposome formulations are especially suitable for aerosol use for delivery to the lungs of patients. Vector DNA and/or virus can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et. al. Gene Ther. 1999, 6:1438-47). Other techniques in formulating expression vectors and virus as therapeutics are found in “DNA-Pharmaceuticals: Formulation and Delivery in Gene Therapy, DNA Vaccination and Immunotherapy” by Martin Schleef (Editor) December 2005, Wiley Publisher, and “Plasmids for Therapy and Vaccination” by Martin Schleef (Editor) May 2001, are incorporated herein as reference. In one embodiment, the dosage for viral vectors is 106 to 1014 viral vector particles per application per patient.

The route of administration, dosage form, and the effective amount vary according to the potency of the IL-17 antagonist, and expression vectors and viral vectors used the gene therapy, and their physicochemical characteristics. The selection of proper dosage is well within the skill of an ordinarily skilled physician.

Gene Therapy

In one embodiment, the IL-17 antagonist is administered to an individual by any one of several gene therapy techniques known to those of skill in the art. In general, gene therapy can be accomplished by either direct transformation of target cells within the mammalian subject (in vivo gene therapy) or transformation of cells in vitro and subsequent implantation of the transformed cells into the mammalian subject (ex vivo gene therapy). A viral vector carries the nucleic acid encoding the IL-17 antagonist (e.g. a soluble form of an IL-17 receptor, an inhibitory anti-IL-17 antibody, an inhibitory anti-IL-17R antibody, a fusion protein containing IL-17 receptor, a fusion protein containing IL-17 binding fragment of IL-17 receptor, an antisense IL-17 nucleic acid molecule, an antisense IL-17 receptor nucleic acid molecule, an siRNA IL-17 nucleic acid molecule, and an siRNA IL-17 receptor nucleic acid molecule) under a tissue specific regulatory element is administered to the individual diagnosed with asthma. The tissue specific regulatory element allows the expression of the IL-17 antagonist in the target cells, for example, the lung epithelial cells. Gene transfer of a soluble IL-17R using an adenoviral vector expressing the soluble IL-17R is described by Li Jianping et. al., 2006, Eur. J. Cardiothorac. Surg. 29: 779-783 and is hereby incorporated by reference.

The principles of gene therapy are disclosed by Oldham, R. K. (In: Principles of Biotherapy, Raven Press, N.Y., 1987), and similar texts. Disclosures of the methods and uses for gene therapy are provided by Boggs, S. S. (Int. J. Cell Clon. 8:80-96 (1990)); Karson, E. M. (Biol. Reprod. 42:39-49 (1990)); Ledley, F. D., In: Biotechnology, A Comprehensive Treatise, volume 7B, Gene Technology, VCH Publishers, Inc. NY, pp 399-458 (1989)), all of which references are incorporated herein by reference.

The nucleic acid encoding the IL-17 antagonist may be introduced into the somatic cells of an animal (particularly mammals including humans) in order to provide a treatment for non-allergen induced asthma. Most preferably, viral or retroviral vectors are employed for as the transfer vehicle this purpose. A suitable vehicle for gene therapy will not promote an immune response to the IL-17 antagonist described herein. The gene therapy virus can be in the form of an adenovirus, adeno-associated virus or lentivirus.

The term “vector”, as used herein, refers to a nucleic acid construct designed for delivery to a host cell or transfer between different host cells.

As used herein, a “retroviral vector” refers to an expression vector that comprises a nucleotide sequence that encodes a transgene and that further comprises nucleotide sequences necessary for packaging of the vector. Preferably, the retroviral transfer vector also comprises the necessary sequences for expressing the transgene in cells.

Retroviral vectors are a common mode of delivery and in this context are retroviruses from which all viral genes have been removed or altered so that no viral proteins are made in cells infected with the vector. Viral replication functions are provided by the use of retrovirus “packaging” cells that produce all of the viral proteins but that do not produce infectious virus.

Introduction of the retroviral vector DNA into packaging cells results in production of virions that carry vector RNA and can infect target cells, but such that no further virus spread occurs after infection. To distinguish this process from a natural virus infection where the virus continues to replicate and spread, the term transduction rather than infection is often used.

In one embodiment, the method described herein provides a recombinant lentivirus for the delivery and expression of a the IL-17 antagonist in either dividing and non-dividing mammalian cells. The HIV-1 based lentivirus can effectively transduce a broader host range than the Moloney Leukemia Virus (MoMLV)-base retroviral systems. Preparation of the recombinant lentivirus can be achieved using the pLenti4/V5-DESTT™, pLenti6/V5-DESTT™ or pLenti vectors together with ViraPower™ Lentiviral Expression systems from Invitrogen.

Examples of use of lentiviral vectors for gene therapy for inherited disorders and various types of cancer, and these references are hereby incorporated by reference (Klein, C. and Baum, C. (2004). Hematol. J., 5, 103-111; Zufferey, R et. al. (1997). Nat. Biotechnol., 15, 871-875; Morizono, K. et. al. (2005). Nat. Med., 11, 346-352; Di Domenico, C. et. al. (2005), Hum.Gene Ther., 16, 81-90; Kim, E. Y., et. al., (2004). Biochem. Biophys. Res. Comm., 318, 381-390).

Non-retroviral vectors also have been used in genetic therapy. One such alternative is the adenovirus (Rosenfeld, M. A., et al., Cell 68:143155 (1992); Jaffe, H. A. et al., Nature Genetics 1:372-378 (1992); Lemarchand, P. et al., Proc. Natl. Acad. Sci. USA 89:6482-6486 (1992)). Major advantages of adenovirus vectors are their potential to carry large segments of DNA (36 Kb genome), a very high titer (10¹¹/ml), ability to infect non-replicating cells, and suitability for infecting tissues in situ, especially in the lung. The most striking use of this vector so far is to deliver a human cystic fibrosis transmembrane conductance regulator (CFTR) gene by intratracheal instillation to airway epithelium in cotton rats (Rosenfeld, M. A., et al., Cell 63:143-155 (1992)). Similarly, herpes viruses may also prove valuable for human gene therapy (Wolfe, J. H. et al., Nature Genetics 1:379-384 (1992)). Of course, any other suitable viral vector may be used for genetic therapy with the present invention.

U.S. Pat. No. 6,531,456 provides methods for the successful transfer of a gene into a solid tumor cell using recombinant AAV virions. Generally, the method described in U.S. Pat. No. 6,531,456 allows for the direct, in vivo injection of recombinant AAV virions into tumor cell masses, e.g., by intra-tumoral injection. The invention also provides for the simultaneous delivery of a second gene using the recombinant AAV virions, wherein the second gene is capable of providing an ancillary therapeutic effect when expressed within the transduced cell. U.S. Pat. No. 6,531,456 is hereby incorporated by reference.

The viron used for gene therapy can be any viron known in the art including but not limited to those derived from adenovirus, adeno-associated virus (AAV), retrovirus, and lentivirus. Recombinant viruses provide a versatile system for gene expression studies and therapeutic applications.

The recombinant AAV virions described above, including the DNA of interest, can be produced using standard methodology, known to those of skill in the art. The methods generally involve the steps of (1) introducing an AAV vector into a host cell; (2) introducing an AAV helper construct into the host cell, where the helper construct includes AAV coding regions capable of being expressed in the host cell to complement AAV helper functions missing from the AAV vector; (3) introducing one or more helper viruses and/or accessory function vectors into the host cell, wherein the helper virus and/or accessory function vectors provide accessory functions capable of supporting efficient recombinant AAV (“rAAV”) virion production in the host cell; and (4) culturing the host cell to produce rAAV virions. The AAV vector, AAV helper construct and the helper virus or accessory function vector(s) can be introduced into the host cell either simultaneously or serially, using standard transfection techniques. Using rAAV vectors, genes can be delivered into a wide range of host cells including many different human and non-human cell lines or tissues. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells(E1-transformed human embryonic kidney cells) or 911 (E1-transformed human embryonic retinal cells) (Human Gene Therapy 7:215-222, 1996). Recombinant adenovirus are generated within the HEK 293 cells.

The use of alternative AAV serotypes other than AAV-2 (Davidson et al (2000), Proc. Natl. Acad. Sci. USA 97(7)3428-32; Passini et al (2003), J. Virol. 77(12):7034-40) has demonstrated different cell tropisms and increased transduction capabilities. With respect to brain cancers, the development of novel injection techniques into the brain, specifically convection enhanced delivery (CED; Bobo et al (1994), Proc. Natl. Acad. Sci. USA 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying a DNA coding sequence for an IL-17 antagonist, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

Pharmaceutical compositions used in the methods described herein can be delivered systemically via in vivo gene therapy. A variety of methods have been developed to accomplish in vivo transformation including mechanical means (e.g., direct injection of nucleic acid into target cells or particle bombardment), recombinant viruses, liposomes, and receptor-mediated endocytosis (RME) (for reviews, see Chang et al. 1994 Gastroenterol. 106:1076-84; Morsy et al. 1993 JAMA 270:2338-45; and Ledley 1992 J. Pediatr. Gastroenterol. Nutr. 14:328-37).

Another gene transfer method for use in humans is the transfer of plasmid DNA in liposomes directly to human cells in situ (Nabel, E. G., et al., Science 249:1285-1288 (1990)). Plasmid DNA should be easy to certify for use in human gene therapy because, unlike retroviral vectors, it can be purified to homogeneity. In addition to liposome-mediated DNA transfer, several other physical DNA transfer methods, such as those targeting the DNA to receptors on cells by conjugating the plasmid DNA to proteins, have shown promise in human gene therapy (Wu, G. Y., et al., J. Biol. Chem. 266:14338-14342 (1991); Curiel, D. T., et al., Proc. Natl. Acad. Sci. USA, 88:8850-8854 (1991)).

For gene therapy viruses, the dosage ranges from 10⁶ to 10¹⁴ particles per application. Alternatively the biolistic gene gun method of delivery may be used. The gene gun is a device for injecting cells with genetic information, originally designed for plant transformation. The payload is an elemental particle of a heavy metal coated with plasmid DNA. This technique is often simply referred to as biolistics. Another instrument that uses biolistics technology is the PDS-1000/He particle delivery system. The proteins, expression vector, and/or gene therapy virus can be coated on minute gold particles, and these coated particles are “shot” into biological tissues such as hemangiomas and melanoma under high pressure. An example of the gene gun-based method is described for DNA based vaccination of cattle by Loehr B. I. et. al. J. Virol. 2000, 74:6077-86.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The present inventions can be defined in any of the following alphabetized paragraphs:
[A] The use of an IL-17 antagonist in the manufacture of medicament for the treatment of non-allergic asthma in an individual.
[B] The use of paragraph [A], wherein the individual is diagnosed with non-allergic asthma.
[C] The use of paragraph [B], wherein the diagnosis is made by measuring the level of neutrophils in a sample of sputum.
[D] The use of paragraph [B] or [C], wherein the non-allergic asthma is ozone-induced asthma.
[E] The use of any of the paragraphs [A]-[D], wherein the IL-17 antagonist is selected from a group consisting of:
   a. a soluble form of an IL-17 receptor;
   b. an inhibitory anti-IL-17 antibody;
   c. an inhibitory anti-IL-17R antibody;
   d. a fusion protein containing IL-17 receptor;
   e. a fusion protein containing IL-17 binding fragment of IL-17 receptor;
   f. an IL-17 antagonistic small molecule;
   g. an antisense IL-17 nucleic acid molecule;
   h. an antisense IL-17 receptor nucleic acid molecule;
   i. an siRNA IL-17 nucleic acid molecule; and
   j. an siRNA IL-17 receptor nucleic acid molecule.
[F] The use of paragraph [E], wherein the medicament further comprise an NKT cell inhibitory agent.
[G] The use of paragraph [F], wherein the agent inhibits NKT cell immune function through interaction with an antigen presenting molecule.
[H] The use of paragraph [E] or [F], wherein the NKT cell inhibitory agent is β-gal actosylceramide.
[I] The use of an IL-17 antagonist in the manufacture of medicament for the treatment of an individual at risk of non-allergic asthma, wherein the individual is determined to be at risk when the level of neutrophils in the sputum of the individual is above a predetermined level.
[J] The use of paragraph [I], wherein the non-allergic asthma is ozone-induced asthma.
[K] The use of paragraph [I] or [J], wherein the IL-17 antagonist is selected from a group consisting of:
   a. a soluble form of an IL-17 receptor;
   b. an inhibitory anti-IL-17 antibody;
   c. an inhibitory anti-IL-17R antibody;
   d. a fusion protein containing IL-17 receptor;
   e. a fusion protein containing IL-17 binding fragment of IL-17 receptor;
   f. an IL-17 antagonistic small molecule;
   g. an antisense IL-17 nucleic acid molecule;
   h. an antisense IL-17 receptor nucleic acid molecule;
   i. an siRNA IL-17 nucleic acid molecule; and
   j. an siRNA IL-17 receptor nucleic acid molecule.
[L] The use of paragraph [K], wherein the medicament further comprise an NKT cell inhibitory agent.
[M] The use of paragraph [L], wherein the agent inhibits NKT cell immune function through interaction with an antigen presenting molecule.
[N] The use of paragraph [L] or [M], wherein the NKT cell inhibitory agent is β-gal actosylceramide.
[O] A method of treating non-allergic asthma in an individual comprising:
   a. selecting an individual diagnosed with non-allergic asthma; and
   b. administering to said individual an effective amount of IL-17 antagonist.
[P] The method of paragraph [O], wherein the diagnosis of non-allergic asthma in said individual is made by measuring the level of neutrophils in a sample of sputum.
[Q] The method of paragraph [O] or [P], wherein the non-allergic asthma is ozone-induced asthma.
[R] The method of paragraph [O] or [Q], wherein the IL-17 antagonist is selected from a group consisting of:
   a. a soluble form of an IL-17 receptor;
   b. an inhibitory anti-IL-17 antibody;
   c. an inhibitory anti-IL-17R antibody;
   d. a fusion protein containing IL-17 receptor;
   e. a fusion protein containing IL-17 binding fragment of IL-17 receptor;
   f. an IL-17 antagonistic small molecule;
   g. an antisense IL-17 nucleic acid molecule;
   h. an antisense IL-17 receptor nucleic acid molecule;
   i. an siRNA IL-17 nucleic acid molecule; and
   j. an siRNA IL-17 receptor nucleic acid molecule.
[S] The method of paragraph [R], further comprising administering to said individual an effective amount of NKT cell inhibitory agent.
[T] The method of paragraph [S], wherein said agent inhibits NKT cell immune function through interaction with an antigen presenting molecule.
[U] The method of paragraph [S] or [T], wherein the NKT cell inhibitory agent is β-galactosylceramide.
[V] A method of treating an individual at risk of non-allergic asthma comprising:
   a. measuring the level of neutrophils in a sample of sputum from an individual;
   b. administering to said individual an effective amount of an IL-17 antagonist when the level of neutrophils is above a predetermined level.
[W] The method of paragraph [V], wherein the non-allergic asthma is ozone-induced asthma.
[X] The method of paragraph [V] or [W], wherein the IL-17 antagonist is selected from a group consisting of:
   a. a soluble form of an IL-17 receptor;
   b. an inhibitory anti-IL-17 antibody;
   c. an inhibitory anti-IL-17R antibody;
   d. a fusion protein containing IL-17 receptor;
   e. a fusion protein containing IL-17 binding fragment of IL-17 receptor;
   f. an IL-17 antagonistic small molecule;
   g. an antisense IL-17 nucleic acid molecule;
   h. an antisense IL-17 receptor nucleic acid molecule;
   i. an siRNA IL-17 nucleic acid molecule; and
   j. an siRNA IL-17 receptor nucleic acid molecule.
[Y] The method of paragraph [X], further comprising administering to said individual an effective amount of an NKT cell inhibitory agent.
[Z] The method of paragraph [Y], wherein the agent inhibits NKT cell immune function through interaction with an antigen presenting molecule.
[AA] The method of paragraph [Y] or [Z], wherein the NKT cell inhibitory agent is β-galactosylceramide.

This invention is further illustrated by the following example which should not be construed as limiting. The contents of all references cited throughout this application, as well as the figures are incorporated herein by reference.

### EXAMPLES

### Material and Methods

Mice—Wild-type BALB/c ByJ and C57BL/6N mice were purchased from The Jackson Laboratory (Bar Harbor, Me.). CD1d^{-/-} and Jα18^{-/-} mice (both backcrossed to BALB/c) were gifts from M. Grusby (Harvard School of Public Health) (50) and M. Taniguchi/T. Nakayama (Chiba University) (51), respectively. IL-4^{-/-}/IL-13^{-/-} double knockout mice were obtained from Andrew McKenzie, Oxford. Female mice were studied at ˜8 wk of age. The Animal Care and Use Committee, Children's Hospital Boston and at Harvard Medical School approved all animal protocols.

Antibodies and Reagents—α-GalactosylCeramide (α-GalCer) was provided by Paul B. Savage (Brigham Young University, Provo Utah), and was used as positive control to induce AHR (data not shown). Neutralizing rat anti-mouse CD1.1 (CD1d) mAb (hybridoma HB323, ATCC, Manassas, Va.) was used as described. Anti-mouse IL-17 blocking Ab (clone eBioTC11-18H10.1) and the control rat IgG1k isotype control Ab (eBioscience, San Diego, Calif.) were used in the blocking experiments.

ELISA—Supernatants of lung cell suspensions were collected 48 h after α-GalCer restimulation (100 ng/ml). Mouse IL-4 and mouse IFN-γ levels were measured by ELISA as previously described (13).

Flow cytometry—Cells were pre-incubated with anti-Fcγ blocking mAb (2.4G2) and washed before staining. Cells were stained with anti-mouse PE-Texas red conjugated CD45, PerCPCy5.5 conjugated CD3, Alexa 750 conjugated CD4 mAb (clone RM4-5). iNKT cells were identified using APC conjugated TCRβ (clone H57-597, eBioscience, San Diego, Calif.) and PE conjugated CD1d:tetramer loaded with PBS57 (NIH, NIAID MHC tetramer core facility, Atlanta, Ga.). For intracellular staining, after permeabilization (Cytofix/cytoperm kit, BD Biosciences, San Jose, Calif.), cells were incubated with FITC conjugated IL-4, FITC conjugated IFN-γ, APC conjugated IL-10, AlexaFluor 647 conjugated IL-17, or the respective isotype control Abs: FITC conjugated rat IgG1k, APC conjugated rat IgG2b or Alexafluor 647 conjugated rat IgG2a (eBioscience, San Diego, Calif.). Cells were collected on a FACS Canto flow cytometer (BD Biosciences, San Jose, Calif.) and analyzed with FlowJo 8.3.3 software (Tree Star Inc., Ashland, Oreg.).

Adoptive transfer of iNKT cells—iNKT cells were purified from splenocytes of wild-type BALB/c or IL-4^{-/-}/IL-13^{-/-}double knockout mice using magnetic cell sorting (MACS), as described (13). Splenic iNKT cells were labeled with PE-conjugated CD1d-tetramer followed by anti-PE microbeads (Miltenyi Biotec, Auburn, Calif.), and then sorted with Auto MACS according to manufacturer's instruction. Purity of iNKT cells was >80%. Purified iNKT cells (1×10⁶) were adoptively transferred into recipient Jα18^{-/-} mice by intravenous injection one day before the first exposure to ozone or air.

Ozone exposure and measurement of airway responsiveness in the ozone model—Mice were placed awake in individual wire mesh cages inside a stainless steel and Plexiglas exposure chamber and exposed to ozone (1 ppm) for 3 h. For room air exposure, a separate and identical exposure chamber was used. Details of the ozone exposure and monitoring have been previously described (52). Exposure was repeated every other day (Days 1, 3 and 5) to optimize airway inflammation and iNKT cell recruitment into the BAL fluid over the shortest period of time, and did not induce any weight changes in the mice (data not shown). As a positive control for lung mechanics assessment, control mice received one intranasal injection of α-GalCer (0.5 μg) the day before the measurement of AHR (data not shown). Twenty-four hours after the last ozone-(Day 6) or α-GalCer-exposure, mice were anesthetized with pentobarbitol (50 mg/kg) and instrumented for the measurement of pulmonary mechanics (BUXCO Electronics, Troy, N.Y.). Mice were tracheostomized, intubated and mechanically ventilated at a tidal volume of 0.2 ml and a frequency of 150 breath/min. as previously described (52). Baseline lung resistance (RL) and responses to aerosolized saline (0.9% NaCl) were first measured, followed by responses to increasing doses of aerosolized acetyl-β-methylcholine chloride (methacholine; MCh) (Sigma-Aldrich, St. Louis, Mo.) (0.32 to 40 mg/ml).

Induction of AHR and measurement of airway responsiveness in the OVA model—To induce AHR, mice were sensitized with 100 μg of OVA (Sigma-Aldrich, St. Louis, Mo.) in alum administered intraperitoneally. After 8 days, mice were exposed to intranasal antigen (50 μg OVA per day) or PBS on 3 consecutive days, as described (13). Responses to inhaled methacholine were assessed in conscious mice placed in a whole-body plethysmograph (Buxco Electronics, Troy, N.Y.) as described (13).

BAL fluid—Following measurement of AHR and euthanasia, the lungs were lavaged twice with 1 ml of PBS 2% FCS and the fluid pooled as described (16). For some experiments, total BAL for each mouse or pooled BAL was stained and analyzed by flow cytometry. BAL iNKT cells numbers were quantified by multiplying hemacytometer cell counts excluding red blood cells by percent of iNKT within a cellular gate that included lymphocytes, monocytes, large epithelial cells and granulocytes (by Forward Scatter and Side Scatter).

Lung tissue—Following measurement of AHR, euthanasia and collection of BAL fluids, lungs were perfused with PBS 2% FCS and portions of lung tissues were treated with collagenase type 4 (Worthington, Lakewood, N.J.). The lymphocyte-enriched fraction was collected at the interface 50%-70% of percoll (Amersham Biosciences, Piscataway, N.J.), and plated for 2 hours with BD GolgiStop (BD Biosciences, San Jose, Calif.) before intracellular staining.

Statistical tests—Differences between groups with parametric distributions were analyzed by Student's t test, otherwise the Mann-Whitney U test was used. Significance for all statistical tests was shown in figures for p<0.05(*) and p<0.01 (**).

### Example 1

### Airway Hyperreactivity Induced by Ozone

NKT Cells are Required for AHR Induced by Repeated Ozone Exposure

Mice were exposed to ozone (1 ppm for 3 h) every other day over a five day period, using a semi-acute protocol that maximized airway inflammatory cell recruitment over a brief period of time. Exposure of wildtype (WT) BALB/c mice to ozone in this manner resulted in higher baseline airway resistance, and in the development of severe AHR (FIG. 1A) and significant airway inflammation, consisting of increased macrophages, lymphocytes, and neutrophils, but not eosinophils, in the broncoalveolar lavage (BAL) fluid (FIG. 1C). Repeated ozone exposure also increased the number of iNKT cells in the BAL fluid by >10 fold (FIG. 1D). To determine the role of these iNKT cells in the development of AHR, WT mice were compared with CD1d^{-/-} mice, which lack the restriction element of NKT cells, and therefore lack NKT cells (6). Surprisingly, CD1^{d-/-} mice exposed to ozone failed to develop AHR (FIG. 1A). The CD1d^{-/-} mice exposed to repeated ozone exposure also resulted in significantly fewer airway macrophages, lymphocytes, and neutrophils in the BAL fluid compared to that of WT mice (FIG. 1C). The requirement for iNKT cells was not dependent of the genetic background of the mice tested (all backcrossed to BALB/c), as the same effects was observed in C57BL/6 mice (data not shown). Pre-treatment of WT mice with an anti-CD1d-blocking mAb to prevent iNKT cell activation also reduced ozone-induced AHR (FIG. 1B), and decreased the number of lymphocytes, neutrophils and iNKT cell numbers in the BAL fluid (FIGS. 1C and 1D). Thus, repeated exposure of mice to ozone over five days induced AHR that depended on the presence of iNKT cells.

Jα18^{-/-} Mice Fail to Develop Ozone-Induced AHR

To confirm that iNKT cells were specifically required for the development of ozone-induced AHR, another iNKT cell deficient strain was examined, Jα18^{-/-} mice, which lack the invariant TCR-α chain, and therefore lack iNKT cells (6). Like CD1d^{-/-} mice, Jα18^{-/-} mice repeatedly exposed to ozone over five days failed to develop AHR, whereas WT mice developed severe AHR (FIG. 2A). Compared to WT mice, Jα18−/− mice exposed to ozone also had significantly reduced numbers of macrophages, lymphocytes, and neutrophils (FIG. 2B) and no detectable iNKT cells (FIG. 2C) in BAL fluid. The specific requirement for iNKT cells in the development of ozone-induced AHR was demonstrated by adoptive transfer of purified WT iNKT cells into Jα18^{-/-} mice, which partially restored the capacity of Jα18^{-/-} mice to develop ozone-induced AHR (FIG. 2D). Full restoration of the AHR response was not achieved by adoptive transfer of WT iNKT cells presumably because insufficient numbers of iNKT cells migrated into the lungs of recipients (the number of iNKT cells in the BAL fluid of recipients was only 30% of that found in WT mice (data not shown)).

IL-17 is Required for Ozone-Induced Neutrophilia and AHR

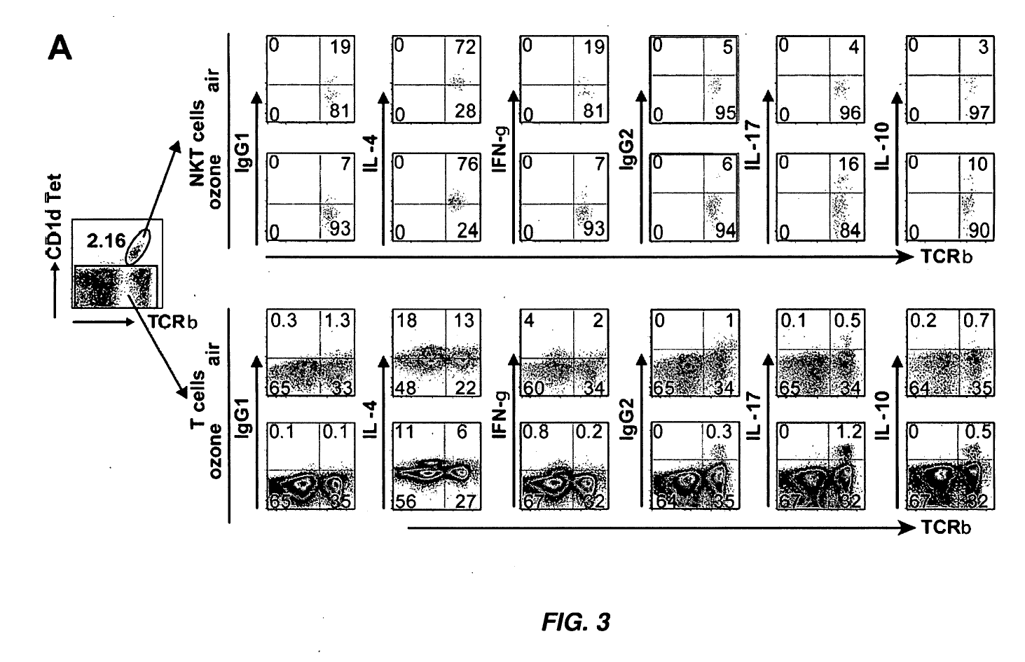

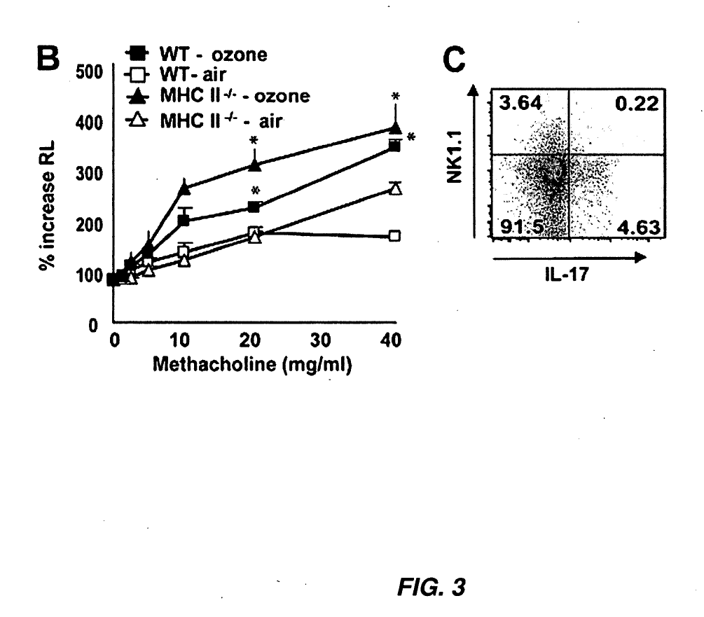

The data indicate that ozone-induced AHR was associated with airway neutrophilia, therefore, the role of IL-17 was evaluated, since IL-17 has been shown to enhance neutrophil migration to sites of inflammation (26, 27). In the present example, iNKT cells and T cells produce IL-17 after ozone exposure. WT BALB/c mice were exposed 3 times to 1 ppm of ozone versus air. Intracellular IL-4, IFN-γ, IL-17 and IL-10 staining was performed on T cell enriched lung cells treated with GolgiStop for 2 hours. Repeated ozone exposure was associated with the induction of IL-17 synthesis in iNKT cells and small number of T cells in the lungs (FIG. 3A), but not in the spleen (data not shown). Note that the total number of cells in the lungs was greatly increased after ozone treatment (3 to 6 fold), and therefore the percentages of cells indicated by flow cytometry underestimated the increases in IL-17 producing cells induced with ozone exposure. In addition, we found that the pulmonary iNKT cells produced IL-4, but not IFN-γ, unlike resting or activated splenic iNKT cells, which produce both IL-4 and IFN-γ, suggesting that IL-4 but not IFN-γ production by iNKT cells also played a role in the induction of ozone-AHR. Ozone also induced IL-10 expression in iNKT cells but not T cells in the lungs (FIG. 3A). To assess expression of NK1.1 by the iNKT cells (not present in BALB/c mice), C57BL/6 mice were exposed to ozone and it was found that the IL-17 producing T cells were NK1.1⁻ (FIG. 3C). This suggests that the IL-17 producing iNKT cells induced with ozone were distinct from the IL-4/IL-13 producing iNKT cells, which are generally NK1.1⁺, at least in C57BL/6 mice.

To determine whether the development of ozone-induced AHR required IL-17 production by conventional CD4+ T cells (Th17 cells) or by iNKT cells, MHC class II−/− mice, which lack conventional CD4+ T cells but have iNKT cells, were exposed to ozone. FIG. 3B shows that the MHC class II−/− mice developed severe ozone-induced AHR and severe airway inflammation (data not shown), just as did the WT mice exposed to ozone. These results clearly indicated that IL-17 producing iNKT cells and not Th17 cells are required for the development of ozone induced AHR, although it is possible that Th17 cells may contribute to the development of AHR.

Ozone-Induced, but Not Allergen-Induced, AHR Requires IL-17

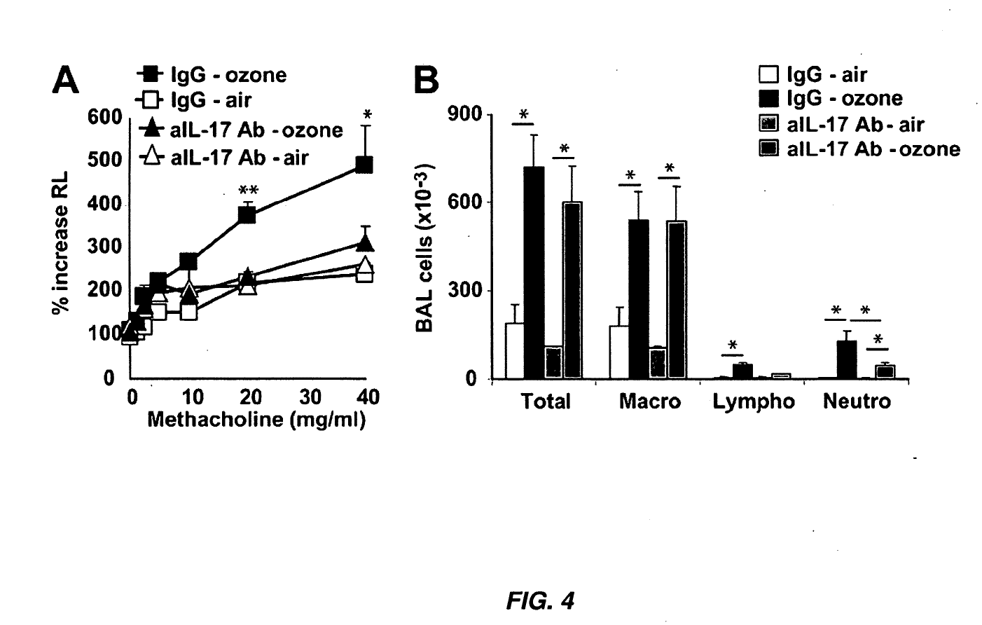

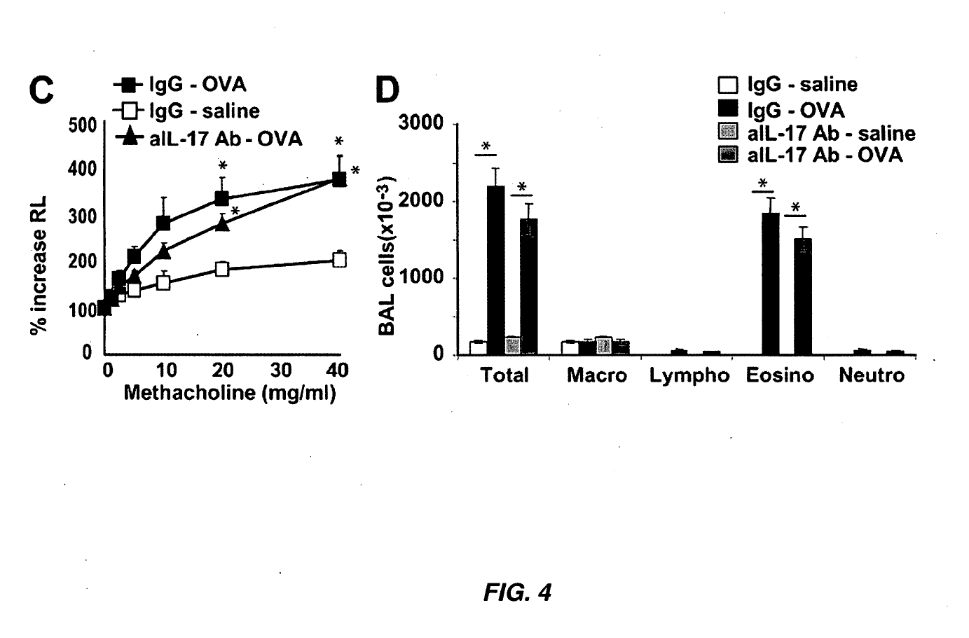

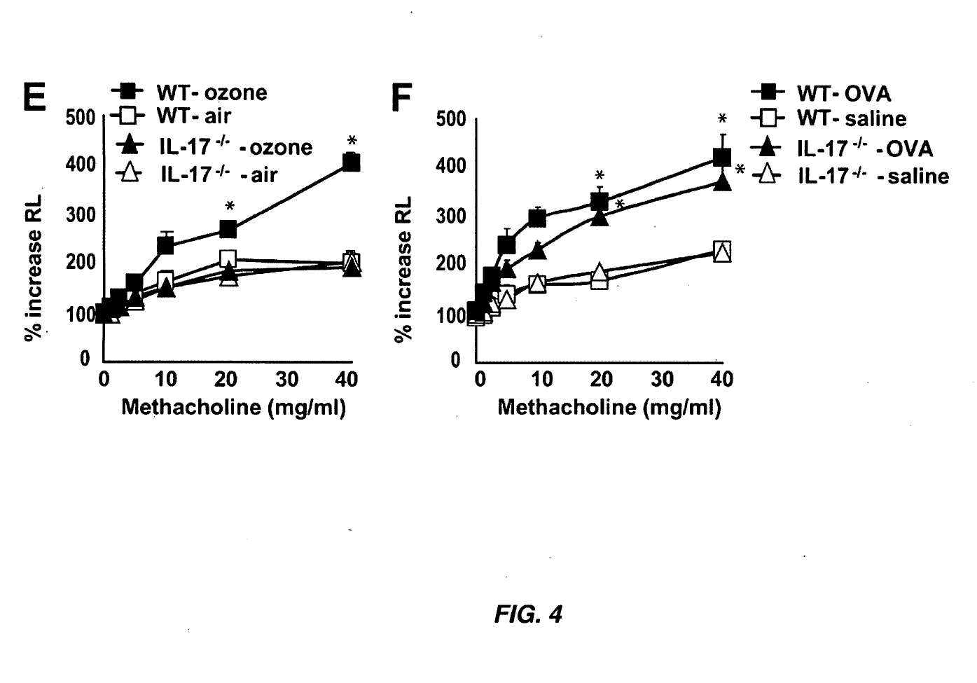

Importantly, IL-17 production in the lungs was absolutely required for ozone-induced AHR, since treatment of mice with an anti-IL-17 blocking mAb greatly reduced ozone-induced AHR (FIG. 4A), airway lymphocytes and neutrophils (but not macrophages) (FIG. 4B). These results were confirmed in IL-17−/− mice, which failed to develop ozone-induced AHR (FIG. 4E) and inflammation. IL-17 production by iNKT cells or T cells was required specifically for ozone-but not allergen-induced AHR, since ovalbumin (OVA)-induced AHR and inflammation occurred normally in WT mice treated with anti-IL-17 blocking mAb (FIGS. 4C and 4D) and in IL-17−/− mice (FIG. 4F). In addition, IL-17 producing cells were not detected in the lungs of mice sensitized and challenged with OVA to induce AHR (data not shown). Therefore, these results demonstrate that IL-17 plays a critical role in the development of AHR and airway inflammation induced by repeated ozone exposure but not by allergen. Thus, while both allergen- and ozone-induced AHR require the presence of iNKT cells, the cytokines produced by these cells are distinct, with IL-17 playing a pathogenic role in ozone-induced, but not allergen-induced AHR.

IL-4 and IL-13 are also involved in ozone-induced AHR and airway inflammation

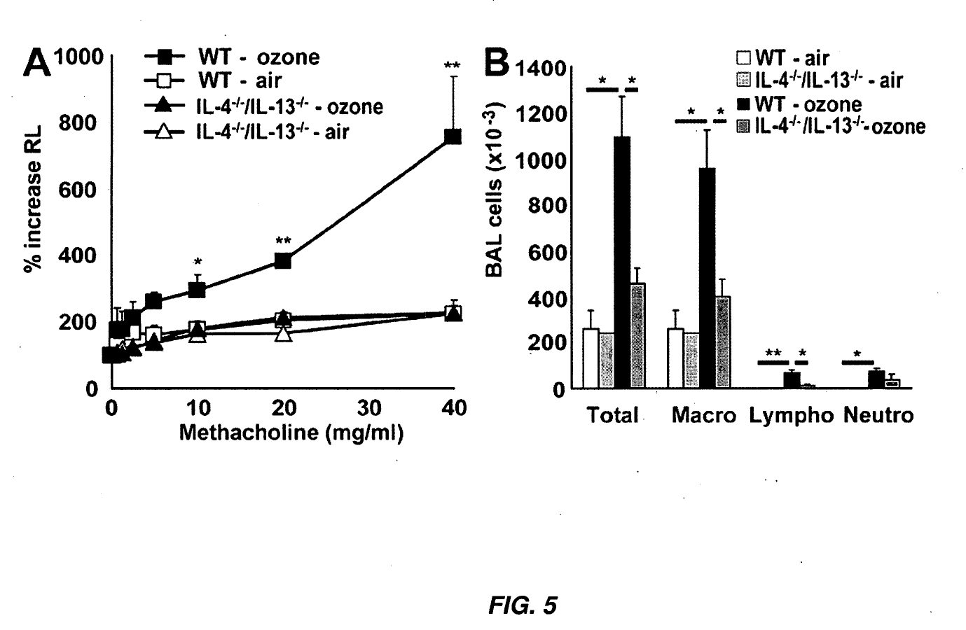

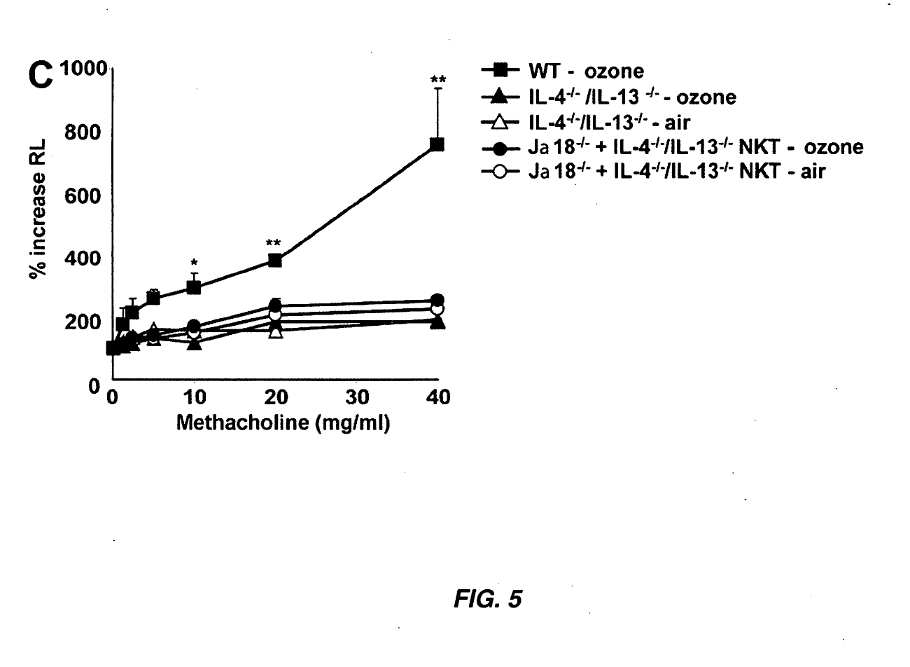

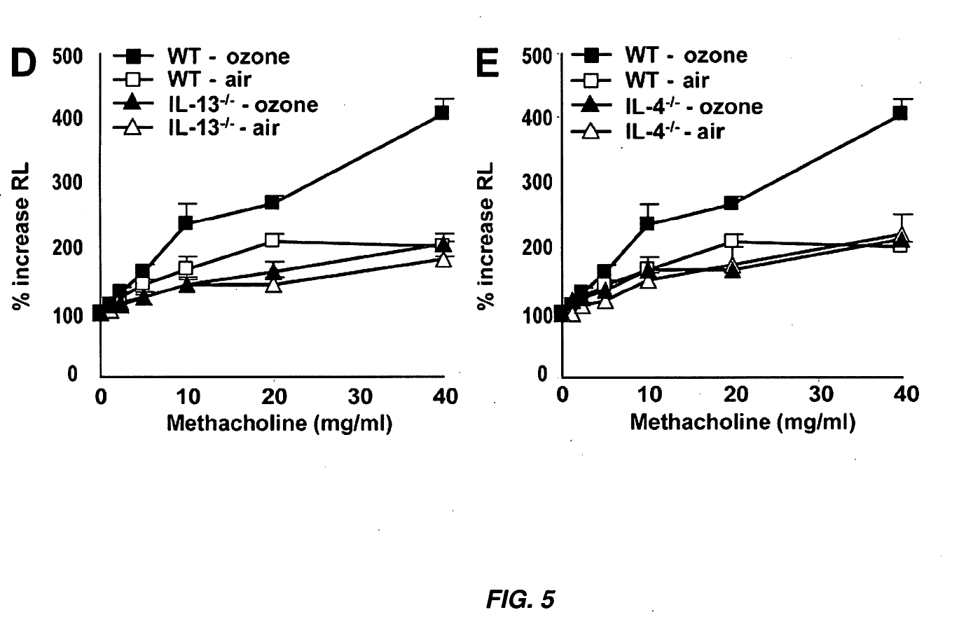

It was previously shown that iNKT cells producing IL-4 and IL-13 are essential in the development of allergen-induced AHR (13). Since IL-4 was present in most of the iNKT cells in the lungs of ozone exposed mice (FIG. 3), experiment was conducted to determine whether IL-4 and IL-13 are also required for AHR induced by repeated ozone exposure. To that end, IL-4^{-/-}/IL-13^{-/-} double knockout mice were exposed to ozone over a 5 day period. After exposure to ozone, the IL-4^{-/-}/IL-13^{-/-} mice failed to develop AHR and had reduced airway inflammation, including significantly fewer macrophages and lymphocytes, compared to WT mice, (FIGS. 5A and 5B). The requirement of IL-4 and/or IL-13 in NKT cells for ozone-induced AHR was confirmed by additional studies demonstrating that adoptive transfer of iNKT cells purified from IL-4^{-/-}/IL-13^{-/-} double knockout mice into Jα18^{-/-} mice failed to reconstitute ozone-induced AHR (FIG. 5C), whereas transfer of WT iNKT cells partially restored AHR (FIG. 2D). Furthermore, IL-13^{-/-} and IL-4^{-/-} mice exposed to ozone both failed to develop AHR (FIGS. 5D and 5E). These results indicate that while allergen- and ozone-induced AHR differ in their requirement for IL-17 and in the types of inflammatory cells present in the airways (eosinophils versus neutrophils), both forms of AHR require iNKT cells producing IL-4 and/or IL-13.

It was previously showed that allergen-induced AHR requires the presence of invariant TCR natural killer T (iNKT) cells, but it is unclear whether ozone-induced AHR had similar requirements. Therefore, wild type BALB/c mice and NKT deficient CD1d^{-/-} or Jα18^{-/-} mice were exposed to ozone (1 ppm ozone for 3 hours every other day over 5 days). Then AHR was analyzed by measuring increases in pulmonary resistance in response to methacholine in anesthetized, intubated and mechanically ventilated mice, and assessed airway inflammation and the recruitment of iNKT cells into the airways. It was found that exposure of wild type BALB/c mice to ozone resulted in significant AHR, and greatly increased the number of iNKT cells in the lungs. Surprisingly, NKT cell deficient CD1d^{-/-} and Jα18^{-/-} mice failed to develop AHR. The failure of Jα18^{-/-} mice to develop AHR was reversed by adoptive transfer of iNKT cells purified from wild type BALB/c mice with CD1d tetramers. Further, treatment of wild type BALB/c mice with anti-CD1d blocking mAb greatly reduced the ozone induced AHR response, confirming the requirement for CD 1d-restricted NKT cells. Moreover, ozone-induced AHR failed to develop in IL4^{-/-}IL13^{-/-} (double knockout) mice, indicating that ozone-induced AHR was dependant on IL-4 and IL-13 production. These results together indicate that ozone induced AHR, like allergen-induced AHR, requires the presence of iNKT cells. Thus ozone-induced and allergen-induced AHR both depend on innate-like immunity, suggesting that a requirement for iNKT cells may unify many different forms of asthma.

In this example, it was demonstrated that repeated exposure to ozone induces AHR mediated by a novel mechanism involving innate-like immunity, iNKT cells and IL-17. Repeated exposure to ozone induces the development of AHR in a process that requires iNKT cells as well as IL-17, but not adaptive immunity, Th2 cells or eosinophils. Ozone exposure was associated with IL-17 production by pulmonary iNKT and T cells, and neutralization of IL-17 prevented the development of ozone-but not allergen-induced AHR.

### Example 2

### Direct Activation of Natural Killer T Cells Induces Airway-Hyperreactivity in Non-Human Primates

The development of allergen-induced airway hyperreactivity (AHR) in mice requires the presence of a novel type of T cell, called invariant Natural Killer T (iNKT) cells (13, 14). iNKT cells represent a distinct lineage of T cells that express characteristics of both conventional T cells and natural killer (NK) cells, and express a highly conserved T cell receptor (TCR) a chain, Vα14-Jα18 in mice, and Vα24-JαQ in human (10). Unlike conventional T cells which recognize protein antigens, iNKT cells recognize glycolipid antigens presented by the non-polymorphic Major Histocompatibility Complex (MHC) class I-like molecule CD1d (10). While a critical role for iNKT cells has been clearly demonstrated in murine models of asthma, it is not yet certain whether iNKT cells play a similar vital role in humans in the development of AHR, a cardinal feature of asthma.

Therefore, the function of iNKT cells in asthma in primates was examined by challenging cynomolgus monkeys with the exquisitely specific, iNKT cell-activating agent, α-GalactocylCeramide (α-GalCer). α-GalCer directly activates iNKT cells by specifically binding to the invariant TCR of iNKT cells in mice and humans (10), and when administered into the lungs causes AHR in wildtype but not in iNKT cell deficient mice (16). Because α-GalCer is extraordinarily specific in directly activating iNKT cells, but not Th2 cells, airway epithelial cells or smooth muscle cells (it has no effects in NKT cell deficient mice), α-GalCer is extremely useful in studying the role of iNKT cells in isolation of Th2 cells. Further, monkeys provide a very useful model for studying human asthma, because monkeys and humans are closely related in terms of their genomes, respiratory physiology and immune responses (52), and since airway challenge of humans with α-GalCer at this time poses unacceptable risks.

Four monkeys were challenged with α-GalCer or vehicle using a cross-over design (approved by animal care and use committees at the Children's Hospital Boston and the Charles River Laboratory), which minimized the effects of genetic variation and the differing past histories in each of the outbred monkeys. In particular, because each monkey served as its own control, the cross-over design allowed us to normalize the different baseline levels of AHR that each monkey possessed, and thus minimized the number of animals required for generating statistically significant results. Each adult cynomolgus monkey was anesthetized, intubated and challenged with nebulized α-GalCer or vehicle. For safety reasons and to avoid any possible toxic effects of α-GalCer that might cause AHR, a dose escalation approach was used, starting with a very low dose of α-GalCer (150 μg/m²), similar to the lowest doses used intravenously in Phase I safety studies in humans with solid tumors. Only one dose of α-GalCer was administered to the monkeys before evaluation of AHR, because following activation with α-GalCer, iNKT cells become anergic temporarily (53). 24 hours after challenge, the monkeys were reintubated and assessed for AHR by measuring lung resistance (RL) in response to increasing concentrations of methacholine.

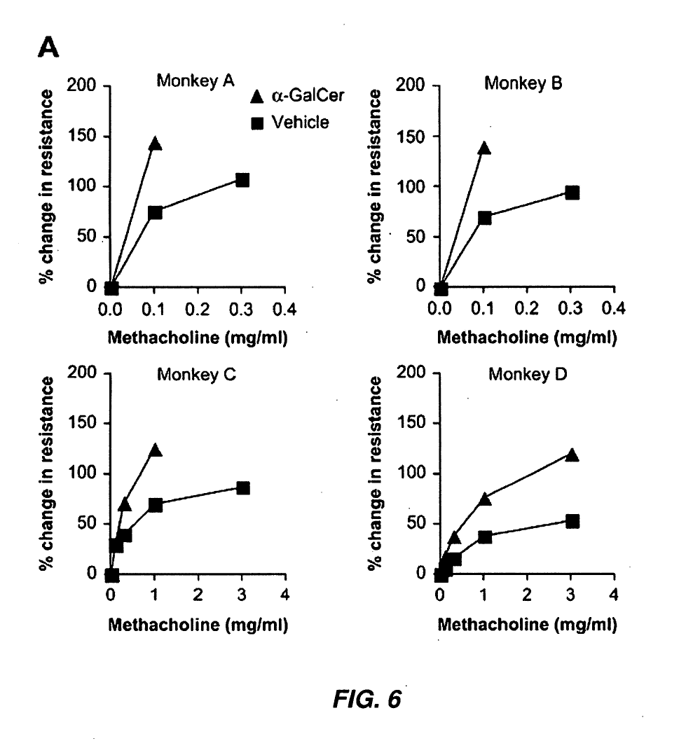

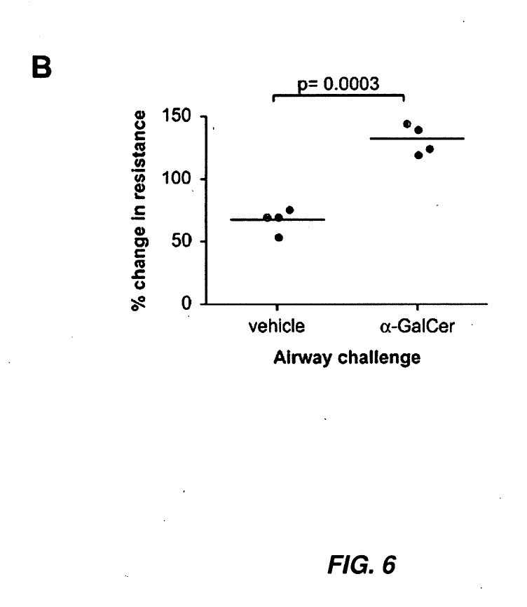

Surprisingly, with the first very low inhaled dose of α-GalCer (150 μg/m²), three of the four monkeys developed significantly increased AHR, compared to challenge with vehicle control (FIG. 6A, monkey A, C, D). The fourth monkey, monkey B, which did not respond to the first dose of α-GalCer, developed a significant AHR response after later receiving a higher dose of α-GalCer (600 μg/m²) (FIG. 6A, monkey B). When combined, data for the four monkeys showed that α-GalCer inhalation induced a significantly higher percentage change in lung resistance (RL) from baseline compared to that with vehicle control (p=0.0003, paired T test comparing results at the highest dose of methacholine used after α-GalCer treatment) (FIG. 6B). While the primary outcome of the study (enhanced AHR in response to methacholine) was achieved, secondary outcomes (increased eosinophils, neutrophils, iNKT cells and IL-4 in the bronchoalveolar lavage (BAL) fluid in response to α-GalCer), while trending towards an increase, did not reach statistical significance, possibly because the dose of α-GalCer was too low due to safety considerations, or due to the small sample size.

Nevertheless, these data clearly demonstrate that direct activation of pulmonary iNKT cells with α-GalCer in monkeys resulted in the development of significant AHR. Furthermore, these results are consistent with observations in mice demonstrating that iNKT cells are required for the development of allergen-induced AHR (13), and that administration of α-GalCer intranasally to naïve mice also results in the development of AHR4. Since α-GalCer functioned to induce AHR without allergen administration, even in class II−/− mice (16), which lack conventional CD4⁺ T cells, these studies with α-GalCer together indicate that iNKT cells can induce AHR even in the absence of Th2 cells. As such, these results suggest that iNKT cells could be important in regulating both allergic and nonallergic forms of asthma. Finally, since treatment outcomes observed in monkeys often predict outcomes that might be observed in humans treated similarly, the results here provide the first functional in vivo evidence in primates that iNKT cells play a critical role in the pathogenesis of human asthma.

The findings presented here in monkeys are also noteworthy, because the role of iNKT cells in human asthma has become controversial, as the particular number of iNKT cells in the lungs has been disputed (53, 54). However, six independent studies have shown that the number of iNKT cells present in the lungs of patients with asthma is greater than that in the lungs of normal individuals (53, 55), although other investigators have been unable to find significant numbers of iNKT cells in the lungs of patients with asthma (54). In this current studies, it was found that the number of iNKT cells in the BAL fluid of monkeys prior to α-GalCer challenge was low (only 0.3-0.6% of the CD3⁺ cells), similar to that in non-asthmatic individuals and in naïve mice. The fact that the administration of α-GalCer induced AHR in monkeys (and in naïve mice) suggests that even small numbers of iNKT cells in the lungs, when activated, can potently induce AHR, and that pulmonary iNKT cells, even when present in small numbers, can indeed be critical effector cells in human asthma.

The results presented here in monkeys are especially important in understanding the pathophysiology of human asthma, not only because of the close relationship between monkeys and humans, but also because iNKT cells, which link innate and adaptive immunity, present a novel and unexpected paradigm in asthma that is difficult to study in humans. iNKT cells express several features of innate immunity, including the capacity to very rapidly produce cytokines on activation, and the expression of a germ line encoded receptor (the invariant TCR) that is remarkably conserved in sequence and function across species (10). This conservation suggests that the invariant TCR of iNKT cells serves as a pattern recognition receptor for glycolipids from pathogens, such as Borrelia burgdorferi, Ehrlichia muris, and Leishmania (10). Furthermore, exogenous glycolipids from pulmonary microbes or from inhaled plant pollens (56), or endogenous glycolipids induced in the lungs by pulmonary inflammation or injury (57) can activate iNKT cells and drive the development of AHR.

The references cited herein and throughout the specification are incorporated herein by reference in their entirety.

### REFERENCES

1 Gold, D. R. & Wright, R. Population disparities in asthma. Annual review of public health 26, 89-113 (2005).
2 Wills-Karp, M. Immunologic basis of antigen-induced airway hyperresponsiveness. Annual review of immunology 17, 255-281 (1999).
3 Voehringer, D., Reese, T. A., Huang, X., Shinkai, K. & Locksley, R. M. Type 2 immunity is controlled by IL-4/IL-13 expression in hematopoietic non-eosinophil cells of the innate immune system. The Journal of experimental medicine 203, 1435-1446 (2006).
4 Gibson, P. G., Simpson, J. L. & Saltos, N. Heterogeneity of airway inflammation in persistent asthma:evidence of neutrophilic inflammation and increased sputum interleukin-8. Chest 119, 1329-1336 (2001).
5 Li, N., et al. Nrf2 is a key transcription factor that regulates antioxidant defense in macrophages and epithelial cells: protecting against the proinflammatory and oxidizing effects of diesel exhaust chemicals. J Immunol 173, 3467-3481 (2004).
6 Kronenberg, M. Toward an understanding of NKT cell biology: progress and paradoxes. Annual review of immunology 23, 877-900 (2005).
7 Brutkiewicz, R. R. CD1d ligands: the good, the bad, and the ugly. J Immunol 177, 769-775 (2006).
8 Kronenberg, M. & Gapin, L. The unconventional lifestyle of NKT cells. Nature reviews 2, 557-568 (2002).
9 Park, S. H., Benlagha, K., Lee, D., Balish, E. & Bendelac, A. Unaltered phenotype, tissue distribution and function of Valpha14(+) NKT cells in germ-free mice. European journal of immunology 30, 620-625 (2000).
10 Bendelac, A., Savage, P. B. & Teyton, L. The biology of NKT cells. Annual review of immunology 25, 297-336 (2007).
11 Mercer, J. C., Ragin, M. J. & August, A. Natural killer T cells: rapid responders controlling immunity and disease. The international journal of biochemistry & cell biology 37, 1337-1343 (2005).
12 Nieuwenhuis, E. E., et al. CD1d-dependent macrophage-mediated clearance of Pseudomonas aeruginosa from lung. Nature medicine 8, 588-593 (2002).
13 Akbari, O., et al. Essential role of NKT cells producing IL-4 and IL-13 in the development of allergen-induced airway hyperreactivity. Nature medicine 9, 582-1588 (2003).
14 Lisbonne, M., et al. Cutting edge: invariant V alpha 14 NKT cells are required for allergen-induced airway inflammation and hyperreactivity in an experimental asthma model. J Immunol 171, 1637-1641 (2003).
15 Smiley, S. T., Kaplan, M. H. & Grusby, M. J. Immunoglobulin E production in the absence of interleukin-4-secreting CD1-dependent cells. Science (New York, N.Y. 275, 977-979 (1997).
16 Meyer, E. H., et al. Glycolipid activation of invariant T cell receptor+ NK T cells is sufficient to induce airway hyperreactivity independent of conventional CD4+ T cells. Proceedings of the National Academy of Sciences of the United States of America 103, 2782-2787 (2006).
17 Akbari, O., et al. CD4+ invariant T-cell-receptor+ natural killer T cells in bronchial asthma. The New England journal of medicine 354, 1117-1129 (2006).
18 Hamzaoui, A., et al. NKT cells in the induced sputum of severe asthmatics. Mediators of inflammation 2006, 71214 (2006).
19 Sen, Y., et al. V alpha 24-invariant NKT cells from patients with allergic asthma express CCR9 at high frequency and induce Th2 bias of CD3+ T cells upon CD226 engagement. J Immunol 175, 4914-4926 (2005).
20 Gent, J. F., et al. Association of low-level ozone and fine particles with respiratory symptoms in children with asthma. Jama 290, 1859-1867 (2003).
21 Babin, S. M., et al. Pediatric patient asthma-related emergency department visits and admissions in Washington, D.C., from 2001-2004, and associations with air quality, socio-economic status and age group. Environ Health 6, 9 (2007).
22 Tolbert, P. E., et al. Air quality and pediatric emergency room visits for asthma in Atlanta, Ga., USA. American journal of epidemiology 151, 798-810 (2000).
23 Aris, R. M., et al. Ozone-induced airway inflammation in human subjects as determined by airway lavage and biopsy. The American review of respiratory disease 148, 1363-1372 (1993).
24 Devlin, R. B., et al. Exposure of humans to ambient levels of ozone for 6.6 hours causes cellular and biochemical changes in the lung. American journal of respiratory cell and molecular biology 4, 72-81 (1991).
25. Bayram, H., Sapsford, R. J., Abdelaziz, M. M. & Khair, O. A. Effect of ozone and nitrogen dioxide on the release of proinflammatory mediators from bronchial epithelial cells of nonatopic nonasthmatic subjects and atopic asthmatic patients in vitro. The Journal of allergy and clinical immunology 107, 287-294 (2001).
25 Ferretti, S., Bonneau, O., Dubois, G. R., Jones, C. E. & Trifilieff, A. IL-17, produced by lymphocytes and neutrophils, is necessary for lipopolysaccharide-induced airway neutrophilia: IL-15 as a possible trigger. J Immunol 170, 2106-2112 (2003).
26 Miyamoto, M., et al. Endogenous IL-17 as a mediator of neutrophil recruitment caused by endotoxin exposure in mouse airways. J Immunol 170, 4665-4672 (2003).
27 Todokoro, M., et al. Effect of ozone exposure on intracellular glutathione redox state in cultured human airway epithelial cells. Inflammation 28, 105-114 (2004).
28 Romieu, I., et al. GSTM1 and GSTP1 and respiratory health in asthmatic children exposed to ozone. Eur Respir J 28, 953-959 (2006).
29 Rangasamy, T., et al. Disruption of Nrf2 enhances susceptibility to severe airway inflammation and asthma in mice. The Journal of experimental medicine 202, 47-159 (2005).
30 Ivanov, I I, et al. The orphan nuclear receptor RORgammat directs the differentiation program of proinflammatory IL-17+ T helper cells. Cell 126, 1121-1133 (2006).
31 Michel, M. L., et al. Identification of an IL-17-producing NK1.1(neg) iNKT cell population involved in airway neutrophilia. The Journal of experimental medicine 204, 995-1001 (2007).
32 Nakae, S., et al. Antigen-specific T cell sensitization is impaired in IL-17-deficient mice, causing suppression of allergic cellular and humoral responses. Immunity 17, 375-387 (2002).
33 Linden, A., Laan, M. & Anderson, G. P. Neutrophils, interleukin-17A and lung disease. Eur Respir J 25, 159-172 (2005).
34 Bullens, D. M., et al. IL-17 mRNA in sputum of asthmatic patients: linking T cell driven inflammation and granulocytic influx? Respiratory research 7, 135 (2006).
35 Kawaguchi, M., et al. IL-17F sequence variant (His161Arg) is associated with protection against asthma and antagonizes wild-type IL-17F activity. The Journal of allergy and clinical immunology 117, 795-801 (2006).
36 Molet, S., et al. IL-17 is increased in asthmatic airways and induces human bronchial fibroblasts to produce cytokines. The Journal of allergy and clinical immunology 108, 430-438 (2001).
37 Hellings, P. W., et al. Interleukin-17 orchestrates the granulocyte influx into airways after allergen inhalation in a mouse model of allergic asthma. American journal of respiratory cell and molecular biology 28, 42-50 (2003).
38 Schnyder-Candrian, S., et al. Interleukin-17 is a negative regulator of established allergic asthma. The Journal of experimental medicine 203, 2715-2725 (2006).
39 D'Amato, G., Liccardi, G., D'Amato, M. & Holgate, S. Environmental risk factors and allergic bronchial asthma. Clin Exp Allergy 35, 1113-1124 (2005).
40 Nel, A. Atmosphere. Air pollution-related illness: effects of particles. Science (New York, N.Y. 308, 804-806 (2005).
41 VanderLaan, P. A., et al. Characterization of the natural killer T-cell response in an adoptive transfer model of atherosclerosis. The American journal of pathology 170, 1100-1107 (2007).
42 Ronmark, E., et al. Obesity increases the risk of incident asthma among adults. Eur Respir J 25, 282-288 (2005).
43 Lu, F. L., et al. Increased pulmonary responses to acute ozone exposure in obese db/db mice. American journal of physiology 290, L856-865 (2006).
44 Cho, H. Y., Zhang, L. Y. & Kleeberger, S. R. Ozone-induced lung inflammation and hyperreactivity are mediated via tumor necrosis factor-alpha receptors. American journal of physiology 280, L537-546 (2001).
45 Johnston, R. A., Schwartzman, I. N., Flynt, L. & Shore, S. A. Role of interleukin-6 in murine airway responses to ozone. American journal of physiology 288, L390-397 (2005).
46 Park, J. W., et al. Interleukin-1 receptor antagonist attenuates airway hyperresponsiveness following exposure to ozone. American journal of respiratory cell and molecular biology 30, 830-836 (2004).
47 Shore, S. A., Schwartzman, I. N., Le Blanc, B., Murthy, G. G. & Doerschuk, C. M. Tumor necrosis factor receptor 2 contributes to ozone-induced airway hyperresponsiveness in mice. American journal of respiratory and critical care medicine 164, 602-607 (2001).
48 Yu, M., Zheng, X., Witschi, H. & Pinkerton, K.E. The role of interleukin-6 in pulmonary inflammation and injury induced by exposure to environmental air pollutants. Toxicol Sci 68, 488-497 (2002).
49 Behar, S. M., Dascher, C. C., Grusby, M.J., Wang, C.R. & Brenner, M.B. Susceptibility of mice deficient in CD1D or TAP1 to infection with Mycobacterium tuberculosis. The Journal of experimental medicine 189, 1973-1980 (1999).
50 Cui, J., et al. Requirement for Valphal4 NKT cells in IL-12-mediated rejection of tumors. Science (New York, N.Y. 278, 1623-1626 (1997).
51 Shore, S. A., Johnston, R. A., Schwartzman, I. N., Chism, D. & Krishna Murthy, G.G. Ozone-induced airway hyperresponsiveness is reduced in immature mice. J Appl Physiol 92, 1019-1028 (2002).
52 Coffman R L., Hessel E M. Nonhuman primate models of asthma. J Exp Med; 201:1875-9 (2005).
53 Akbari O, Faul J L, Hoyte E G, Berry G J, Wahlstrom J, Kronenberg M, et al. CD4+ invariant T-cell-receptor+ natural killer T cells in bronchial asthma. N Engl J Med; 354:1117-29 (2006).
54 Vijayanand P, Seumois G, Pickard C, Powell R M, Angco G, Sammut D, et al. Invariant natural killer T cells in asthma and chronic obstructive pulmonary disease. N Engl J Med; 356:1410-22 (2007).
55 Meyer E H, Dekruyff R H, Umetsu D T. T Cells and NKT Cells in the Pathogenesis of Asthma. Annu Rev Med (2007).
56 Agea E, Russano A, Bistoni O, Mannucci R, Nicoletti 1, Corazzi L, et al. Human CD1-restricted T cell recognition of lipids from pollens. J Exp Med; 202:295-308 (2005).
57 Pichavant M, Goya S, Meyer E H, Johnston R A, Kim H Y, Matangkasombut P, et al. Ozone exposure in a mouse model induces airway hyperreactivity that requires the presence of Natural Killer T cells and IL-17. J Exp Med. (2008) In press.

1-14. (canceled)

15. A method of treating non-allergic asthma in an individual comprising:
a. selecting an individual diagnosed with non-allergic asthma; and
b. administering to said individual an effective amount of IL-17 antagonist.

16. The method of claim 15, wherein the diagnosis of non-allergic asthma in said individual is made by measuring the level of neutrophils in a sample of sputum.

17. The method of claim 15, wherein the non-allergic asthma is ozone-induced asthma.

18. The method of claim 15, wherein the IL-17 antagonist is selected from a group consisting of:
a. a soluble form of an IL-17 receptor;
b. an inhibitory anti-IL-17 antibody;
c. an inhibitory anti-IL-17R antibody;
d. a fusion protein containing IL-17 receptor;
e. a fusion protein containing IL-17 binding fragment of IL-17 receptor;
f. an IL-17 antagonistic small molecule;
g. an antisense IL-17 nucleic acid molecule;
h. an antisense IL-17 receptor nucleic acid molecule;
i. an siRNA IL-17 nucleic acid molecule; and
j. an siRNA IL-17 receptor nucleic acid molecule.

19. The method of claim 18, further comprising administering to said individual an effective amount of NKT cell inhibitory agent.

20. The method of claim 19, wherein said agent inhibits NKT cell immune function through interaction with an antigen presenting molecule.

21. The method of claim 19, wherein the NKT cell inhibitory agent is β-galactosylceramide.

22. A method of treating an individual at risk of non-allergic asthma comprising:
a. measuring the level of neutrophils in a sample of sputum from an individual;
b. administering to said individual an effective amount of an IL-17 antagonist when the level of neutrophils is above a predetermined level.

23. The method of claim 22, wherein the non-allergic asthma is ozone-induced asthma.

24. The method of claim 22, wherein the IL-17 antagonist is selected from a group consisting of:
a. a soluble form of an IL-17 receptor;
b. an inhibitory anti-IL-17 antibody;
c. an inhibitory anti-IL-17R antibody;
d. a fusion protein containing IL-17 receptor;
e. a fusion protein containing IL-17 binding fragment of IL-17 receptor;
f. an IL-17 antagonistic small molecule;
g. an antisense IL-17 nucleic acid molecule;
h. an antisense IL-17 receptor nucleic acid molecule;
i. an siRNA IL-17 nucleic acid molecule; and
j. an siRNA IL-17 receptor nucleic acid molecule.

25. The method of claim 24, wherein further comprising administering to said individual an effective amount of an NKT cell inhibitory agent.

26. The method of claim 25, wherein the agent inhibits NKT cell immune function through interaction with an antigen presenting molecule.

27. The method of claim 25, wherein the NKT cell inhibitory agent is β-galactosylceramide.

28. The method of claim 21, wherein the NKT cell inhibitory agent is β-galactosylceramide.

29. The method of claim 26, wherein the NKT cell inhibitory agent is β-galactosylceramide.


SEQUENCE LISTING (6 sequences)

SEQ ID NO: 1 - DNA, 3429 bp, Homo sapiens
ctgggcccgggctggaagccggaagcgagcaaagtggagccgactcgaactccaccgcggaaaagaaagcctcagaacgttcgttcgctgcgtccccagccggggccgagccctccgcgacgccagccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaa

SEQ ID NO: 2 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 3 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 4 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 5 - protein, 6 aa, Artificial Sequence
  source: /note=“Description of Artificial Sequence Synthetic 6x His tag”
HHHHHH

SEQ ID NO: 6 - DNA, 23 bp, Artificial Sequence
  source: /note=“Description of artificial sequence Synthetic oligonucleotide”
aannnnnnnnnnnnnnnnnnntt